(19) United States
(12) Patent Application Publication
(10) Pub. No.: US 2014/0248218 A1
(43) Pub. Date: Sep. 4, 2014

(54) ROS-SENSITIVE FLUORESCENT PROBES

(71) Applicant: The Regents of the University of California, Oakland, CA (US)

(72) Inventors: Kevin B. Daniel, San Diego, CA (US); Seth M. Cohen, Carlsbad, CA (US); Jody L. Major Jourden, Atlanta, GA (US)

(73) Assignee: The Regents of the University of California, Oakland, CA (US)

(21) Appl. No.: 14/175,380

(22) Filed: Feb. 7, 2014

Related U.S. Application Data
(60) Provisional application No. 61/762,706, filed on Feb. 8, 2013.

(51) Int. Cl. G01N 33/58; A61K 49/00

(52) U.S. Cl. CPC G01N 33/582; A61K 49/0039; A61K 49/0041
USPC 424/9.6; 549/213; 549/223; 549/289; 436/135; 435/34

(57) ABSTRACT

Herein are provided, inter alia, compositions including boronic esters, which in the presence of H₂O₂, provide for the detection of ROS compounds such as endogenous H₂O₂ and methods of using the compositions to detect ROS compounds.

### CROSS-REFERENCES TO RELATED APPLICATIONS

This application claims priority to U.S. Provisional Application No. 61/762,706, Filed Feb. 7, 2013.

### STATEMENT AS TO RIGHTS TO INVENTIONS MADE UNDER FEDERALLY SPONSORED RESEARCH AND DEVELOPMENT

This invention was made with Government support under grant numbers DK007233 and GM098435 awarded by the National Institutes of Health. The Government has certain rights in the invention.

### BACKGROUND OF THE INVENTION

Reactive oxygen species (ROS) encompass a wide array of endogenously produced intermediates that directly result from oxygen metabolism in biological systems.^{[1]} The chemical biology of ROS, especially hydrogen peroxide (H₂O₂), is rather complex, as recent studies show that controlled generation of H₂O₂ is necessary to maintain cellular functions such as growth, proliferation, and immune system function.^{[2]} However, misregulation in the production of ROS can lead to significant oxidative damage due to the inability of cells to effectively manage oxidation-reduction equilibrium.^{[1,3]} It is the specific cellular localization and concentration that alter the role of ROS from one of cell signaling to that of oxidative stress and disease.^{[2a,2e]} H₂O₂ is a major ROS byproduct and has been studied as a common indicator for oxidative stress in a number of pathologies including cancer,^{[4]} cardiovascular^{[5]} and neurodegenerative^{[6]} diseases, and diabetes.^{[7]} There is a need in the art to understand the roles and implications of H₂O₂ generation in biological systems and for probes to detect physiological levels of H₂O₂. Provided herein are solutions to these and other problems in the art.

### BRIEF SUMMARY OF THE INVENTION

Disclosed herein, inter alia, are compositions of boronic acid-based and boronic ester-based probes that are sensitive to ROS compounds, including H₂O₂. The compositions include a compound having the formula:

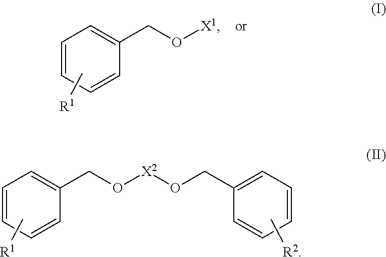

R¹ and R² of compounds (I) and (II) are independently —B(OH)₂ or

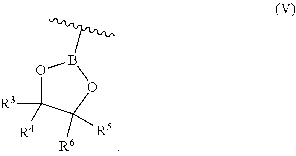

R³, R⁴, R⁵ and R⁶ of the compound of formula (V) are independently hydrogen, halogen, —N₃, —NO₂, —CF₃, —CCl₃, —CBr₃, —CI₃, —CN, —OH, —NH₂, —COOH, —CONH₂, —SH, —SO₃H, —SO₄H, —SO₂NH₂, —NHNH₂, —ONH₂, —OCH₃, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. X¹ is a monovalent pro-fluorophore moiety. X² is a divalent pro-fluorophore moiety.

Also disclosed herein, inter alia, are methods of using the probes. A method is provided for detecting a ROS compound. The method includes contacting a compound of formula (I) or formula (II) with a ROS compound. The ROS compound is allowed to react with the compound of formula (I) and remove R¹. The ROS compound is allowed to react with the compound of formula (II) and remove R¹ and R². Removing R¹ of the compound of formula (I) or R¹ and R² of the compound of formula (II) forms a fluorescent compound. The fluorescent compound is detected thus detecting the ROS compound.

Also provided is a method for detecting a ROS compound in vivo in a subject. The method includes administering to a subject, a compound of formula (I) or formula (II). The compound of formula (I) is allowed to react with a ROS compound remove R¹. The compound of formula (II) is allowed to react with a ROS compound and remove R¹ and R². Removing R¹ of the compound of formula (I) or R¹ and R² of the compound of formula (II) forms a fluorescent compound. The fluorescent compound is detected thus detecting the ROS compound in vivo in the subject.

### BRIEF DESCRIPTION OF THE DRAWINGS

FIG. 1. Prior art design of boronic ester-based H₂O₂-sensitive fluorescent probes.

FIG. 2. Synthesis of boronic ester pro-fluorophores described herein using an exemplary derivatized fluorescein compound (FBBBE) with a H₂O₂-sensitive boronic ester trigger appended via a benzyl ether linkage (top). Scheme of deprotection and fluorescence activation of FBBBE in the presence of H₂O₂ (bottom).

FIG. 3. Structures of H₂O₂-sensitive pro-fluorophores FBBBE and CBBE, along with their corresponding non-fluorescent controls, FBn and CBn.

FIG. 4. Fluorescent response of 50 μM FBBBE to H₂O₂ (5 eq, 250 μM): the dotted line represents the initial spectrum and subsequent spectra were recorded every 4 min after the addition of H₂O₂— the insert shows the fluorescent response of 10 μM FBBBE to various concentrations of added H₂O₂ (Spectra were collected after incubation with H₂O₂ at room temperature for 15 min. and the collected emission was integrated between 500-700 nm (λexc=480 nm) and the spectra were acquired in 50 mM HEPES (pH 7.5)).

FIG. 5. Molecular imaging of H₂O₂ in RAW 264.7 cells: Confocal fluorescence image of cells treated with (a) PBS and 50 μM FBBBE (b) 1 μg/mL LPS for 24 h and 50 μM FBBBE (c) 100 μM H₂O₂ for 1 h and 50 μM FBBBE (d) PBS and 50 μM FBn (e) 1 μg/mL LPS for 24 h and 50 μM FBn (f) 100 μM H₂O₂ for 1 h and 50 μM FBn.

FIG. 6. Confocal fluorescence images of H₂O₂ in mouse brain treated with FBBBE for 1 h: Brain tissue treated with PBS (a-c) or 100 μM H₂O₂ (d-f); 50 μM FBBBE (a, d); images nuclear staining with DAPI (4′,6-diamidino-2-phenylindole, blue) (b, e); overlay of the FBn and DAPI staining (c, f).

FIG. 7. Absorption spectra of FBBBE (50 μM) in 50 mM HEPES (pH 7.5) in the presence of H₂O₂ (100 eq, 5 mM) monitored every 10 min for 5 h: the dotted line is the initial spectrum (an authentic sample of fluorescein (50 μM) in 50 mM HEPES (pH 7.5) is shown); the absorption spectra of FBBE (50 μM) in 50 mM HEPES (pH 7.5).

FIG. 8. Fluorescence emission of FBn (50 μM) in 50 mM HEPES (pH 7.5) in response to H₂O₂: the initial spectrum is shown below and the line above, almost superimposed, shows the emission spectrum after 1 h incubation H2O2 (9 eq, 450 μM) (λexc=480 nm).

FIG. 9. Absorption spectra of FBBE (50 μM) in 50 mM HEPES (pH 7.5) in the presence of H₂O₂ (18 eq, 900 μM) monitored every 2 min for 20 min: the dotted line is the initial spectrum and an authentic sample of fluorescein (50 μM) in 50 mM HEPES (pH 7.5) is shown.

FIG. 10. Fluorescent response of FBBBE (5 μM) in 50 mM HEPES (pH 7.5) to H₂O₂: the dotted line represents the initial spectrum and subsequent spectra were recorded every 4 min after the addition of H2O2 (4 eq, 25 μM) (λexc=370 nm).

FIG. 11. Fluorescence response of 5 μM CBBE to increasing concentrations of added H₂O₂: Spectra were collected after incubation of CBBE with H2O2 at RT for 15 min (the collected emission was integrated between 410 and 700 nm (λexc=370 nm) and all spectra were acquired in 50 mM HEPES (pH 7.5)).

FIG. 12. Absorption spectra of CBBE (50 μM) in 50 mM HEPES (pH 7.5) in the presence of H₂O₂ (9 eq, 450 μM) monitored every 2 min for 1 h: the dotted line is the initial spectra and an authentic sample of 7-hydroxycoumarin (50 μM) in 50 mM HEPES (pH 7.5) is shown.

FIG. 13. Fluorescence emission of CBn (50 μM) in 50 mM HEPES (pH 7.5): the initial spectrum is shown in black and the coincident line shows the emission spectra after 1 h incubation with H₂O₂ (9 eq, 450 μM) (λexc=370 nm).

FIG. 14. Fluorescence turn-on of FBBBE (1 μM) in 50 mM HEPES (pH 7.5): the bottom line represents the initial spectrum and the top line is the final spectrum after 1.5 h incubation with H₂O₂ (300 eq, 300 μM) (λexc=480 nm).

FIG. 15. Fluorescence turn-on of CBBE (1 μM) in 50 mM HEPES (pH 7.5): the bottom line represents the initial spectrum and the top line is the final spectrum after 30 min incubation with H₂O₂ (300 eq, 300 μM) (λexc=370 nm).

FIG. 16. Histograms of fluorescent responses of 1 μM FBBBE to hydrogen peroxide (H₂O₂) and hypochlorite (OCl⁻): (Data shown are for 100 μM of each ROS) hydrogen peroxide and hypochlorite were delivered from 30% and 5% aqueous solutions, respectively and spectra were acquired in 50 mM HEPES (pH 7.5) wherein all data were obtained after incubation with the appropriate ROS at 25° C.; the collected emission was integrated between and 700 nm (λₑₓ=480 nm), and bars represent relative responses (left to right) at 5 (white), 15 (light gray), 30 (gray), 45 (dark gray), and 60 min (black) after addition of ROS.

FIG. 17. Quantitative analysis of FBBBE and FBn turn-on response in RAW 264.7 cells using ImageJ: analysis of mean fluorescence of triplicate measurements of FBBBE and FBn in PBS, H₂O₂, and LPS treated RAW cells calculated using two-way ANOVA (GraphPad Prism) where the mean fluorescence within each group (FBBBE and FBn) is statistically significant between the different treatments (p<0.001) and the mean fluorescence between each group (FBBBE and FBn) is also statistically significant (p<0.001).

FIG. 18. Quantitative analysis of FBBBE and FBn turn-on response in brain tissue using ImageJ: analysis of mean fluorescence of triplicate measurements of FBBBE and FBn in PBS and H₂O₂ treated brain tissue calculated using two-way ANOVA (GraphPad Prism) where the mean fluorescence within each group (FBBBE and FBn) is not significant between the different treatments and the mean fluorescence between each group (FBBBE and FBn) is statistically significant (p<0.001).

FIG. 19. Confocal fluorescence images of H₂O₂ in mouse brain treated with FBn for 1 h. Brain tissue treated with (a-c) PBS (d-f) 100 μM H₂O₂; 50 μM FBn (a, d); nuclear staining with DAPI (4′,6-diamidino-2-phenylindole, blue) (b, e); overlay of the FBn and DAPI staining (c, f) (All confocal images were obtained at 80× magnification).

FIG. 20. Confocal fluorescence images of H₂O₂ in mouse spinal cord treated with FBBBE for 1 h: spinal cord treated with (a-c) PBS (d-f) 100 μM H₂O₂; 50 μM FBBBE (a, d); nuclear staining with DAPI (4′,6-diamidino-2-phenylindole) (b, e); overlay of the FBBBE and DAPI staining (c, f) (All confocal images were obtained at 40× magnification).

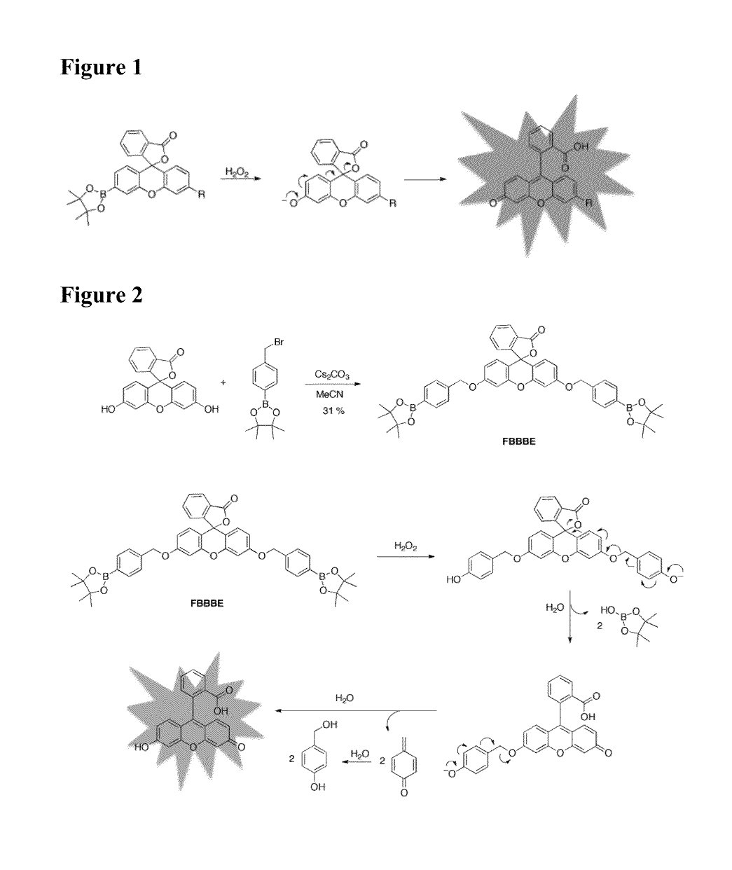

### DETAILED DESCRIPTION OF THE INVENTION

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain, or combination thereof, which may be fully saturated, mono- or polyunsaturated and can include di- and multivalent radicals, having the number of carbon atoms designated (i.e., C₁-C₁₀ means one to ten carbons). An “alkyl” is not cyclized. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or combinations thereof, consisting of at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. A heteroalkyl is not cyclized. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two heteroatoms may be consecutive, such as, for example, —CH₂—NH—OCH₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, refers to a moiety with formula, —C(O)R, where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain one or more heteroatoms selected from N, O, and S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. A 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

The term “oxo,” as used herein, means an oxygen that is double bonded to a carbon atom.

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R—SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN, and —NO₂ in a number ranging from zero to (2m′+1), where m′ is the total number of carbon atoms in such radical. R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include boron (B), oxygen (O), nitrogen (N), sulfur (S), phosphorus (P), and silicon (Si).

A “substituent group,” as used herein, means a group selected from the following moieties:
(A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from: oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

The term “acceptable salts” is meant to include salts of the compounds disclosed herein that are prepared with acids or bases, depending on the particular substituents found on the compounds. When compounds of the present invention contain relatively acidic functionalities, base addition salts can be obtained by contacting the neutral form of such compounds with a sufficient amount of the desired base, either neat or in a suitable inert solvent. Examples of acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

Thus, the compounds of the present invention may exist as salts, such as with acceptable acids or bases. The present invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, tautomers, geometric isomers, and individual isomers are encompassed within the scope of the present invention. The compounds of the present invention do not include those that are known in the art to be too unstable to synthesize and/or isolate.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (⁴C). All isotopic variations of the compounds of the present invention, whether radioactive or not, are encompassed within the scope of the present invention.

The symbol “ [symbol] ” denotes the point of attachment of a chemical moiety to the remainder of a molecule or chemical formula.

A “reactive oxygen species” or “ROS” as used herein generally refers to free radicals, reactive anions containing oxygen atoms, or molecules containing oxygen atoms that can either produce free radicals or are chemically activated by them. Accordingly, reactive oxygen species may include, without limitation, superoxide radicals, hydrogen peroxide, peroxynitrite (e.g. ONOO⁻), lipid peroxides, hydroxyl radicals, thiyl radicals, superoxide anion, organic hydroperoxide, RO. alkoxy and ROO. peroxy radicals, and hypochlorite (OCl⁻). One of the main sources of reactive oxygen species (ROS) in vivo is aerobic respiration, although reactive oxygen species may also be produced by peroxisomal b-oxidation of fatty acids, microsomal cytochrome P450 metabolism of xenobiotic compounds, stimulation of phagocytosis by pathogens or lipopolysacchrides, arginine metabolism, tissue specific enzymes. Accumulating oxidative damage may also affect the efficiency of mitochondria and further increase the rate of ROS production.

A “pro-fluorophore moiety,” “monovalent pro-fluorophore,” “divalent pro-fluorophore,” “trivalent pro-fluorophore,” or “tetravalent pro-fluorophore,” refers to a substituent that has suppressed fluorescence when attached to the remainder of the compounds described herein relative to the fluorescence of the compound formed from the substituent when detached from the remainder of the compounds described herein. Thus, a “pro-fluorophore moiety” may be a fluorophore (fluorescent compound) in its monovalent, divalent, trivalent or tetravalent form attached to the remainder of the compounds described herein. In embodiments, the compound described herein attached to a pro-fluorophore moiety is reacted with a ROS as described herein to form a fluorescent compound (e.g. a fluorophore). Fluorophores may be detected using techniques known in the art. The terms “monovalent,” “divalent,” “trivalent,” and “tetravalent” refer to radicals derived from a fluorophore that can form one, two, three, or four bonds respectively. Fluorophores contemplated herein include but are not limited to fluorescein and its conjugates, derivatives, and analogues, including those described herein; coumarin and its conjugates, derivatives, and analogues including those described herein; dansyl, bimane, eosin, rhodamine, cyanines, nile red, xanthones, xanthenes, flazo-orange, Snarf1, resorufin, and conjugates, derivatives, and analogues thereof.

The term “fluorescein” as used herein refers to fluorescent derivatization agents. As used herein, fluorescein includes its derivatives such as, for example, fluorescein isothiocyanate (e.g. FITC), carboxyfluorescsein, succinimidyl esters of carboxyfluorescein (e.g. FAM, 6-JOE)), fluorescein-X-succinimidyl esters (e.g. SFX), and fluorescein dichlorotriazine (e.g. DTAF). In embodiments, fluorescein has the formula:

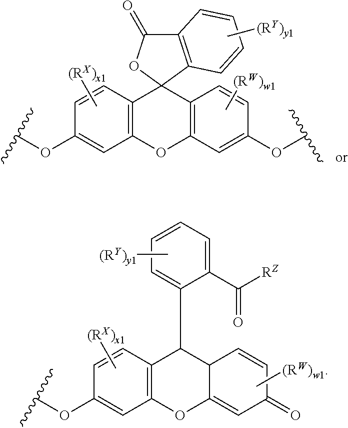

R^{W} and R^{X} are independently hydrogen, halogen, —N₃, CF₃, —CCl₃, —CBr₃, —CI₃, CN, —CHO, —OH, —OCH₃, NH₂, COOH, —CONH₂, NO₂, SH, —SO₂, —SO₂Cl, —SO₃H, —SO₄H, —SO₂NH₂, —NHNH₂, —ONH₂, —NHC(O)NHNH₂, unsubstituted C₁-C₁₀ alkyl, unsubstituted 2 to 10 membered heteroalkyl, unsubstituted 3 to 6 membered cycloalkyl, unsubstituted 3 to 6 membered heterocycloalkyl, unsubstituted 5 or 6 membered aryl, or unsubstituted 5 or 6 membered heteroalkyl. w1 and x1 are independently 1, 2, or 3. In embodiments, R^{W} and R^{X} are independently hydrogen, halogen or —OCH₃.

R^{Y} is independently, hydrogen, halogen, —N₃, CF₃, —CCl₃, —CBr₃, —CI₃, —CN, —CHO, —OH, —OCH₃, NH₂, COOH, —CONH₂, NO₂, SH, —SO₂, —SO₂Cl, —SO₃H, —SO₄H, —SO₂NH₂, —NHNH₂, —ONH₂, —NHC(O)NHNH₂, unsubstituted C₁-C₁₀ alkyl, unsubstituted 2 to 10 membered heteroalkyl, unsubstituted 3 to 6 membered cycloalkyl, unsubstituted 3 to 6 membered heterocycloalkyl, unsubstituted 5 or 6 membered aryl, or unsubstituted 5 or 6 membered heteroalkyl. y1 is 1, 2, 3, or 4. In embodiments, R^{Y} is independently hydrogen, halogen, —COOH, —CH₂Br, Iodoacetamido, malemide, N-succinimidyl ester, N-hydroxysuccinimidyl ester, hexanoic acid, hexanoic acid N-hydroxysuccinimide ester, or isothiocyanate.

R^{Z} is hydrogen, halogen, —N₃, —CF₃, —CCl₃, —CBr₃, —CI₃, —CN, —CHO, —OH, —OCH₃, —NH₂, COOH, —CONH₂, NO₂, SH, —SO₂, —SO₂Cl, —SO₃H, —SO₄H, —SO₂NH₂, —NHNH₂, —ONH₂, —NHC(O)NHNH₂, unsubstituted C₁-C₁₀ alkyl, unsubstituted 2 to 10 membered heteroalkyl, unsubstituted 3 to 6 membered cycloalkyl, unsubstituted 3 to 6 membered heterocycloalkyl, unsubstituted 5 or 6 membered aryl, or unsubstituted 5 or 6 membered heteroalkyl. In embodiments, R^{Z} is hydrogen, Iodoacetamido, N-succinimidyl ester, hexanoic acid, hexanoic acid N-hydroxysuccinimide ester, or isothiocyanate.

The term “coumarin” as used herein refers to fluorescent derivatization agents. As used herein, coumarin includes its derivatives such as, for example, halo-substituted coumarins (e.g. Chlorocoumarin, fluorocoumarin, bromocoumarin and its derivatives), hydroxycoumarin and its derivatives including umbelliferone and its derivatives, cyanocoumarin and its derivatives, methylcoumarin and its derivatives, ethoxycoumarin and its derivatives, benzocoumarin and its derivatives, phenylcoumarin and its derivatives, acetylcoumarin and its derivatives, and carboxylated derivatives and succinimidyl esters thereof. In embodiments, the coumarin has the formula:

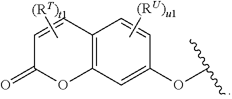

R^{T} and R^{U} are independently hydrogen, halogen, —N₃, CF₃, —CCl₃, —CBr₃, —CI₃, CN, —CHO, —OH, —OCH₃, NH₂, COOH, —CONH₂, NO₂, SH, —SO₂, —SO₂Cl, —SO₃H, —SO₄H, —SO₂NH₂, —NHNH₂, —ONH₂, —NHC(O)NHNH₂, unsubstituted C₁-C₁₀ alkyl, unsubstituted 2 to 10 membered heteroalkyl, unsubstituted 3 to 6 membered cycloalkyl, unsubstituted 3 to 6 membered heterocycloalkyl, unsubstituted 5 or 6 membered aryl, or unsubstituted 5 or 6 membered heteroalkyl. t1 is 1 or 2. u1 is 1, 2, or 3. In embodiments, R^{T} is hydrogen, halogen, —NH₂, —NO₂, —OH, —CN, —COOH, —CF₃, —OCH₃, —OCH₂CH₃, —CH₂Br, —CH₂COOH, substituted or unsubstituted alkyl, substituted or unsubstituted heteroalkyl, or substituted or unsubstituted aryl. In embodiments, R^{T} is formyl, methyl, ethyl, propyl, bromoacetal, acetyl, acetamide, methoxymethyl, allyloxy, propanoic acid, or succinimidyl ester. In embodiments, R^{U} is hydrogen, halogen, —OH, —NH₂, —CH₃, —NO₂, -EtNH₃, methoxy, acetyl, allyloxy, ethoxy, or acetic acid.

“Analog,” “analogue,” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

Conjugates described herein may be synthesized using bioconjugate or conjugate chemistry. Conjugate chemistry includes coupling two molecules together to form an adduct. Conjugation may be a covalent modification. Currently favored classes of conjugate chemistry reactions available with reactive known reactive groups are those which proceed under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

Useful reactive functional groups used for conjugate chemistries herein include, for example:

(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;

(b) hydroxyl groups which can be converted to esters, ethers, aldehydes, etc.

(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;

(d) dienophile groups which are capable of participating in Diels-Alder reactions such as, for example, maleimido groups;

(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;

(f) sulfonyl halide groups for subsequent reaction with amines, for example, to form sulfonamides;

(g) thiol groups, which can be converted to disulfides, reacted with acyl halides, or bonded to metals such as gold;

(h) amine or sulfhydryl groups, which can be, for example, acylated, alkylated or oxidized;

(i) alkenes, which can undergo, for example, cycloadditions, acylation, Michael addition, etc;

(j) epoxides, which can react with, for example, amines and hydroxyl compounds;

(k) phosphoramidites and other standard functional groups useful in nucleic acid synthesis;

(l) metal silicon oxide bonding; and

(m) metal bonding to reactive phosphorus groups (e.g. phosphines) to form, for example, phosphate diester bonds.

(n) azides coupled to alkynes using copper catalyzed cycloaddition click chemistry.

The reactive functional groups can be chosen such that they do not participate in, or interfere with, the chemical stability of the conjugate described herein. Alternatively, a reactive functional group can be protected from participating in the crosslinking reaction by the presence of a protecting group.

Molecular imaging of H₂O₂ with reaction-based fluorescent probes represents a non-invasive approach to monitor the chemistry of a particular ROS (e.g. H₂O₂) in living systems. The specific spatial and temporal distribution of H₂O₂ may be analyzed within a cell or a tissue (e.g. organ). Several selective probes have been reported for the detection of a number of ROS including nitric oxide,^{[8]} peroxynitrite,^{[9]} superoxide,^{[10]} singlet oxygen^{[11]} and H₂O₂^{[11]}. However, these probes fail to address visualization of nominal levels of H₂O₂ typically found in biological systems and may have solubility issues in aqueous solvents. Furthermore, no straightforward synthetic route exists for these probes.

Herein are disclosed novel boronic esters which in the presence of H₂O₂, provide for the detection of endogenous H₂O₂ and a one-step, highly accessible synthetic procedure that allows for gram scale preparation using routine synthetic techniques.

When designing a fluorescent probe for biological imaging, several key factors should be considered including: synthetic ease, aqueous solubility and stability, kinetic rates of deprotection, and fluorescent turn-on response (i.e. conversion from pro-fluorophore to fluorophore). Ideally, a probe should also have the ability to image biologically relevant levels of H₂O₂ with or without external stimulation. Current H₂O₂ probes fail to meet these factors as these probes detect H₂O₂ only after exogenous addition, or by adding H₂O₂ stimulants such as phorbol myristate acetate (PMA) or epidermal growth factor receptor (EGFr). Thus, while these probes can detect exogenous or upregulated levels of H₂O₂ (such as in oxidative-mediated cell signaling), they are unable to detect nominal levels of H₂O₂, including H₂O₂ concentrations in biological systems. The compounds herein provide for detecting of H₂O₂ at nominal levels, including H₂O₂ concentrations typically found in biological systems (e.g. about 10 μM to about 100 μM H₂O₂).

Provided herein are compounds having the formula:

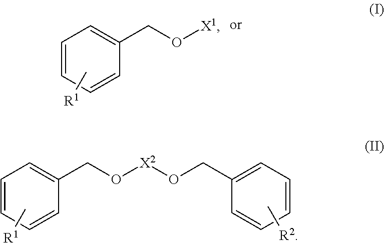

In the compound of formula (I) or formula (II), R¹ and R² are independently —B(OH)₂ or

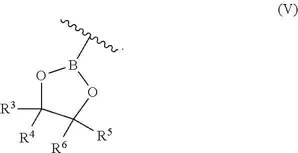

R³, R⁴, R⁵ and R⁶ of the compound of formula (V) are independently hydrogen, halogen, —N₃, —NO₂, —CF₃, —CCl₃, —CBr₃, —CI₃, —CN, —OH, —NH₂, —COOH, —CONH₂, —SH, —SO₃H, —SO₄H, —SO₂NH₂, —NHNH₂, —ONH₂, —OCH₃, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. X¹ is a monovalent pro-fluorophore moiety. X² is a divalent pro-fluorophore moiety.

R¹ and R² may independently be —B(OH)₂. In embodiments, R¹ is —B(OH)₂. In embodiments, R² is —B(OH)₂. In embodiments, R¹ and R² are —B(OH)₂. In embodiments R¹ and R² are independently

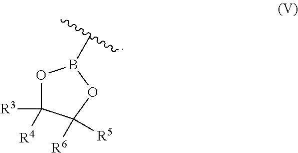

In embodiments, R¹ and R² are the same (e.g. formula (V) for R¹ and R² is identical). In embodiments, R¹ and R² are different (e.g. formula (V) for R¹ and R² is different). In embodiments, at least one of R¹ and R² in the compound of formula (II) is a moiety having formula (V).

R³, R⁴, R⁵ and R⁶ of the compound of formula (V) may independently be hydrogen, halogen, —N₃, —NO₂, —CF₃, —CCl₃, —CBr₃, —CI₃, —CN, —OH, —NH₂, —COOH, —CONH₂, —SH, —SO₃H, —SO₄H, —SO₂NH₂, —NHNH₂, —ONH₂, —OCH₃, or —NHC(O)NHNH₂. R³, R⁴, R⁵ and R⁶ may independently be hydrogen, halogen, —N₃, —NO₂, —CF₃, —CCl₃, —CBr₃, —CI₃, —CN, —OH, —NH₂, —COOH, —CONH₂, —SH, —OCH₃.

R³, R⁴, R⁵ and R⁶ of the compound of formula (V) may independently be hydrogen, halogen, —N₃, —NO₂, —CH₃, —CF₃, —CCl₃, —CBr₃, —CI₃, —CN, —OH, —NH₂, —COOH, —CONH₂, —SH, —SO₃H, —SO₄H, —SO₂NH₂, —NHNH₂, —ONH₂, —OCH₃, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R³, R⁴, R⁵ and R⁶ of the compound of formula (V) may independently be hydrogen, halogen, —N₃, —NO₂, —CH₃, —CF₃, —CCl₃, —CBr₃, —CI₃, —CN, —OH, —NH₂, —COOH, —CONH₂, —SH, —SO₃H, —SO₄H, —SO₂NH₂, —NHNH₂, —ONH₂, —OCH₃, or —NHC(O)NHNH₂. R³, R⁴, R⁵ and R⁶ may independently be hydrogen, halogen, —N₃, —NO₂, —CH₃, —CF₃, —CCl₃, —CBr₃, —CI₃, —CN, —OH, —NH₂, —COOH, —CONH₂, —SH, —OCH₃.

In embodiments, at least one of R³, R⁴, R⁵ and R⁶ is an electron withdrawing group (e.g. a group that removes electron density from the moiety to which it is attached). In embodiments, none of R³, R⁴, R⁵ and R⁶ is an electron withdrawing group. In embodiments, at least one of R³, R⁴, R⁵ and R⁶ is hydrogen. In embodiments, R³, R⁴, R⁵ and R⁶ are hydrogen. In embodiments, R³ is not hydrogen. In embodiments, R⁴ is not hydrogen. In embodiments, R⁵ is not hydrogen. In embodiments, R⁶ is not hydrogen. In embodiments, none of R³, R⁴, R⁵ and R⁶ is hydrogen.

R³, R⁴, R⁵ and R⁶ may independently be substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R³, R⁴, R⁵ and R⁶ may independently be substituted or unsubstituted alkyl. R³, R⁴, R⁵ and R⁶ may independently be R⁷-substituted or unsubstituted alkyl. R³, R⁴, R⁵ and R⁶ may independently be substituted or unsubstituted C₁-C₂₀ alkyl. R³, R⁴, R⁵ and R⁶ may independently be R⁷-substituted or unsubstituted C₁-C₂₀ alkyl. R³, R⁴, R⁵ and R⁶ may independently be substituted or unsubstituted C₁-C₁₀ alkyl. R³, R⁴, R⁵ and R⁶ may independently be R⁷-substituted or unsubstituted C₁-C₁₀ alkyl. R³, R⁴, R⁵ and R⁶ may independently be substituted or unsubstituted C₁-C₅ alkyl. R³, R⁴, R⁵ and R⁶ may independently be R⁷-substituted or unsubstituted C₁-C₅ alkyl. In embodiments, at least one of R³, R⁴, R⁵ and R⁶ is R⁷-substituted or unsubstituted C₁-C₅ alkyl.

In embodiments, R³, R⁴, R⁵ and R⁶ are independently hydrogen, methyl, substituted or unsubstituted ethyl, or substituted or unsubstituted propyl. In embodiments, R³, R⁴, R⁵ and R⁶ are independently methyl, substituted or unsubstituted ethyl, or substituted or unsubstituted propyl. In embodiments, R³, R⁴, R⁵ and R⁶ are independently hydrogen, methyl, R⁷-substituted or unsubstituted ethyl, or R⁷-substituted or unsubstituted propyl. In embodiments, R³, R⁴, R⁵ and R⁶ are independently methyl, R⁷-substituted or unsubstituted ethyl, or R⁷-substituted or unsubstituted propyl.

R³, R⁴, R⁵ and R⁶ may independently be hydrogen or methyl. R³, R⁴, R⁵ and R⁶ may independently be methyl. R³, R⁴, R⁵ and R⁶ may independently be hydrogen or unsubstituted ethyl. R³, R⁴, R⁵ and R⁶ may independently be unsubstituted ethyl. R³, R⁴, R⁵ and R⁶ may independently be hydrogen or unsubstituted propyl. R³, R⁴, R⁵ and R⁶ may independently be unsubstituted propyl. In embodiments, at least one of R³, R⁴, R⁵ and R⁶ is methyl. In embodiments, R³, R⁴, R⁵ and R⁶ are methyl.

In embodiments, R³, R⁴, R⁵ and R⁶ are independently hydrogen or substituted or unsubstituted ethyl. R³, R⁴, R⁵ and R⁶ may independently be substituted or unsubstituted ethyl. R³, R⁴, R⁵ and R⁶ may independently be hydrogen or R⁷-substituted or unsubstituted ethyl. R³, R⁴, R⁵ and R⁶ may independently be R⁷-substituted or unsubstituted ethyl.

R³, R⁴, R⁵ and R⁶ may independently be hydrogen or substituted or unsubstituted propyl. R³, R⁴, R⁵ and R⁶ may independently be substituted or unsubstituted propyl. R³, R⁴, R⁵ and R⁶ may independently be hydrogen or R⁷-substituted or unsubstituted propyl. R³, R⁴, R⁵ and R⁶ may independently be R⁷-substituted or unsubstituted propyl.

R³, R⁴, R⁵ and R⁶ may independently be substituted or unsubstituted heteroalkyl. R³, R⁴, R⁵ and R⁶ may independently be R⁷-substituted or unsubstituted heteroalkyl. R³, R⁴, R⁵ and R⁶ may independently be substituted or unsubstituted 2 to 20 membered heteroalkyl. R³, R⁴, R⁵ and R⁶ may independently be R⁷-substituted or unsubstituted 2 to 20 membered heteroalkyl. R³, R⁴, R⁵ and R⁶ may independently be substituted or unsubstituted 2 to 10 membered heteroalkyl. R³, R⁴, R⁵ and R⁶ may independently be R⁷-substituted or unsubstituted 2 to 10 membered heteroalkyl. R³, R⁴, R⁵ and R⁶ may independently be substituted or unsubstituted 2 to 6 membered heteroalkyl. R³, R⁴, R⁵ and R⁶ may independently be R⁷-substituted or unsubstituted 2 to 6 membered heteroalkyl.

R³, R⁴, R⁵ and R⁶ may independently be substituted or unsubstituted cycloalkyl. R³, R⁴, R⁵ and R⁶ may independently be R⁷-substituted or unsubstituted cycloalkyl. R³, R⁴, R⁵ and R⁶ may independently be substituted or unsubstituted 3 to 20 membered cycloalkyl. R³, R⁴, R⁵ and R⁶ may independently be R⁷-substituted or unsubstituted 3 to 20 membered cycloalkyl. R³, R⁴, R⁵ and R⁶ may independently be substituted or unsubstituted 3 to 10 membered cycloalkyl. R³, R⁴, R⁵ and R⁶ may independently be R⁷-substituted or unsubstituted 3 to 10 membered cycloalkyl. R³, R⁴, R⁵ and R⁶ may independently be substituted or unsubstituted 3 to 6 membered cycloalkyl. R³, R⁴, R⁵ and R⁶ may independently be R⁷-substituted or unsubstituted 3 to 6 membered cycloalkyl.

R³, R⁴, R⁵ and R⁶ may independently be substituted or unsubstituted heterocycloalkyl. R³, R⁴, R⁵ and R⁶ may independently be R⁷-substituted or unsubstituted heterocycloalkyl. R³, R⁴, R⁵ and R⁶ may independently be substituted or unsubstituted 3 to 20 membered heterocycloalkyl. R³, R⁴, R⁵ and R⁶ may independently be R⁷-substituted or unsubstituted 3 to 20 membered heterocycloalkyl. R³, R⁴, R⁵ and R⁶ may independently be substituted or unsubstituted 3 to 10 membered heterocycloalkyl. R³, R⁴, R⁵ and R⁶ may independently be R⁷-substituted or unsubstituted 3 to 10 membered heterocycloalkyl. R³, R⁴, R⁵ and R⁶ may independently be substituted or unsubstituted 3 to 6 membered heterocycloalkyl. R³, R⁴, R⁵ and R⁶ may independently be R⁷-substituted or unsubstituted 3 to 6 membered heterocycloalkyl.

R³, R⁴, R⁵ and R⁶ may independently be substituted or unsubstituted aryl. R³, R⁴, R⁵ and R⁶ may independently be R⁷-substituted or unsubstituted aryl. R³, R⁴, R⁵ and R⁶ may independently be substituted or unsubstituted 5 to 10 membered aryl. R³, R⁴, R⁵ and R⁶ may independently be R⁷-substituted or unsubstituted 5 to 10 membered aryl. R³, R⁴, R⁵ and R⁶ may independently be substituted or unsubstituted 5 to 8 membered aryl. R³, R⁴, R⁵ and R⁶ may independently be R⁷-substituted or unsubstituted 5 to 8 membered aryl. R³, R⁴, R⁵ and R⁶ may independently be substituted or unsubstituted 5 or 6 membered aryl. R³, R⁴, R⁵ and R⁶ may independently be R⁷-substituted or unsubstituted 5 or 6 membered aryl.

R³, R⁴, R⁵ and R⁶ may independently be substituted or unsubstituted heteroaryl. R³, R⁴, R⁵ and R⁶ may independently be R⁷-substituted or unsubstituted heteroaryl. R³, R⁴, R⁵ and R⁶ may independently be substituted or unsubstituted 5 to 10 membered heteroaryl. R³, R⁴, R⁵ and R⁶ may independently be R⁷-substituted or unsubstituted 5 to 10 membered heteroaryl. R³, R⁴, R⁵ and R⁶ may independently be substituted or unsubstituted 5 to 8 membered heteroaryl. R³, R⁴, R⁵ and R⁶ may independently be R⁷-substituted or unsubstituted 5 to 8 membered heteroaryl. R³, R⁴, R⁵ and R⁶ may independently be substituted or unsubstituted 5 or 6 membered heteroaryl. R³, R⁴, R⁵ and R⁶ may independently be R⁷-substituted or unsubstituted 5 or 6 membered heteroaryl.

R⁷ is halogen, —N₃, —NO₂, —CF₃, —CCl₃, —CBr₃, —CI₃, —CN, —OH, —NH₂, —COOH, —CONH₂, —SH, —SO₃H, —SO₄H, —SO₂NH₂, —NHNH₂, —ONH₂, —OCH₃, —NHC(O)NHNH₂, R⁸-substituted or unsubstituted alkyl, R⁸-substituted or unsubstituted heteroalkyl, R⁸-substituted or unsubstituted cycloalkyl, R⁸-substituted or unsubstituted heterocycloalkyl, R⁸-substituted or unsubstituted aryl, or R⁸-substituted or unsubstituted heteroaryl. In embodiments, R⁷ is halogen, —NO₂, —CF₃, —CN, —OH, —NH₂, —COOH, —CONH₂, —SH, —OCH₃, or R⁸-substituted or unsubstituted alkyl. In embodiments, R⁷ is halogen, —CF₃, —OH, —NH₂, —COOH, —CONH₂, or R⁸-substituted or unsubstituted alkyl. In embodiments, R⁷ is R⁸-substituted or unsubstituted alkyl, R⁸-substituted or unsubstituted heteroalkyl, R⁸-substituted or unsubstituted cycloalkyl, R⁸-substituted or unsubstituted heterocycloalkyl, R⁸-substituted or unsubstituted aryl, or R⁸-substituted or unsubstituted heteroaryl. In embodiments, R⁷ is R⁸-substituted or unsubstituted alkyl.

R⁸ is halogen, —N₃, —NO₂, —CF₃, —CCl₃, —CBr₃, —CI₃, —CN, —OH, —NH₂, —COOH, —CONH₂, —SH, —SO₃H, —SO₄H, —SO₂NH₂, —NHNH₂, —ONH₂, —OCH₃, —NHC(O)NHNH₂, R⁹-substituted or unsubstituted alkyl, R⁹-substituted or unsubstituted heteroalkyl, R⁹-substituted or unsubstituted cycloalkyl, R⁹-substituted or unsubstituted heterocycloalkyl, R⁹-substituted or unsubstituted aryl, or R⁹-substituted or unsubstituted heteroaryl. In embodiments, R⁸ is halogen, —N₃, —NO₂, —CF₃, —CCl₃, —CBr₃, —CI₃, —CN, —OH, —NH₂, —COOH, —CONH₂, —SH, —SO₃H, —SO₄H, —SO₂NH₂, —NHNH₂, —ONH₂, —OCH₃, —NHC(O)NHNH₂. In embodiments, R⁸ is halogen, —NO₂, —CF₃, —CN, —OH, —NH₂, —COOH, —CONH₂, or —OCH₃. In embodiments, R⁷ is R⁸-substituted or unsubstituted alkyl,

where R⁸ is halogen, —NO₂, —CF₃, —CN, —OH, —NH₂, —COOH, —CONH₂, —OCH₃, or R⁹-substituted or unsubstituted alkyl, R⁹-substituted or unsubstituted heteroalkyl, R⁹-substituted or unsubstituted cycloalkyl, R⁹-substituted or unsubstituted heterocycloalkyl, R⁹-substituted or unsubstituted aryl, or R⁹-substituted or unsubstituted heteroaryl. In embodiments, R⁷ is R⁸-substituted or unsubstituted alkyl, where R⁸ is halogen, —CF₃, —OH, —NH₂, —COOH, —CONH₂, —OCH₃.

R⁹ is halogen, —N₃, —NO₂, —CF₃, —CCl₃, —CBr₃, —CI₃, —CN, —OH, —NH₂, —COOH, —CONH₂, —SH, —SO₃H, —SO₄H, —SO₂NH₂, —NHNH₂, —ONH₂, —OCH₃, —NHC(O)NHNH₂, unsubstituted alkyl, unsubstituted heteroalkyl, unsubstituted cycloalkyl, unsubstituted heterocycloalkyl, unsubstituted aryl, or unsubstituted heteroaryl.

X¹ may be a monovalent pro-fluorescein, monovalent pro-coumarin, monovalent pro-dansyl, monovalent pro-bimane, monovalent pro-BODIPY, monovalent pro-eosin, monovalent pro-rhodamine, monovalent pro-texas red, monovalent pro-PyMPO, monovalent pro-cyanine; monovalent pro-nile red, monovalent pro-xanthone (e.g. substituted or unsubstituted xanthone), monovalent-pro-xanthene (e.g. substituted or unsubstituted xanthene), monovalent pro-flazo orange, monovalent pro-Snarf1, or a monovalent pro-resorufin, or conjugates, derivatives, or analogues thereof.

In embodiments, monovalent pro-fluorescein (including conjugates, derivatives, and analogues thereof), monovalent pro-coumarin (including conjugates, derivatives, and analogues thereof), monovalent pro-dansyl, monovalent pro-bimane, monovalent pro-eosin, monovalent pro-rhodamine, monovalent pro-cyanine, monovalent nile red, monovalent xanthone, monovalent xanthene, monovalent flazo orange, monovalent Snarf1, or monovalent resorufin, including conjugates, derivatives and analogues thereof.

In embodiments, X¹ is monovalent pro-fluorescein, monovalent pro-coumarin, monovalent pro-dansyl, monovalent pro-bimane, monovalent pro-BODIPY, monovalent pro-eosin, monovalent pro-rhodamine, monovalent pro-texas red, monovalent pro-PyMPO, or monovalent pro-cyanine, including conjugates, derivatives, and analogues thereof. X¹ may be a monovalent pro-fluorescein (including conjugates, derivatives, and analogues thereof) or a monovalent pro-coumarin (including conjugates, derivatives, and analogues thereof). X¹ may be a monovalent pro-fluorescein (including conjugates, derivatives, and analogues thereof). X¹ may be a monovalent pro-coumarin (including conjugates, derivatives, and analogues thereof).

In embodiments, X² is divalent pro-fluorescein, divalent pro-coumarin, divalent pro-dansyl, divalent pro-bimane, divalent pro-texas red, divalent pro-PyMPO, divalent pro-BODIPY, divalent pro-eosin, divalent pro-rhodamine, or divalent pro-cyanine, divalent nile red, divalent xanthones, divalent substituted xanthenes, divalent flazo orange, divalent Snarf1, or divalent resorufin, including conjugates, derivatives, and analogues thereof. X² may be a divalent pro-fluorescein (including conjugates, derivatives, and analogues thereof) or a divalent pro-coumarin (including conjugates, derivatives, and analogues thereof). X² may be a divalent pro-fluorescein (including conjugates, derivatives, and analogues thereof). X² may be a divalent pro-coumarin (including conjugates, derivatives, and analogues thereof).

In embodiments, the compound of formula (I) has the formula:

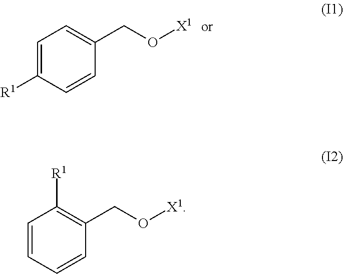

R¹ and X¹ are as described herein. In embodiments, R¹ is a moiety having formula (V), where R³, R⁴, R⁵ and R⁶ are independently C₁-C₅ alkyl. X¹ may be a monovalent pro-fluorescein (including conjugates, derivatives, and analogues thereof) or a monovalent pro-coumarin (including conjugates, derivatives, and analogues thereof).

In embodiments, the compound of formula (I) has the formula:

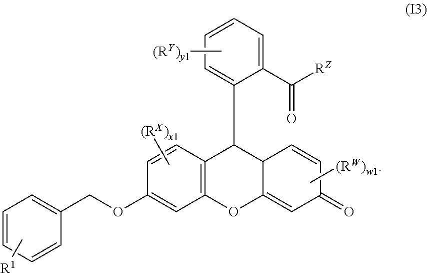

R¹, R^{W}, R^{X}, R^{Y}, R^{Z}, w1, x1, and y1 are as described herein. In embodiments, R^{W} and R^{X} are independently hydrogen, halogen or —OCH₃. In embodiments, R^{Y} and R^{Z} are independently hydrogen, halogen, —OH, —OCH₃, —COOH, —CH₂Br, Iodoacetamido, N-succinimidyl ester, hexanoic acid, hexanoic acid N-hydroxysuccinimide ester, or isothiocyanate.

In embodiments, the compound of formula (I3) has the formula:

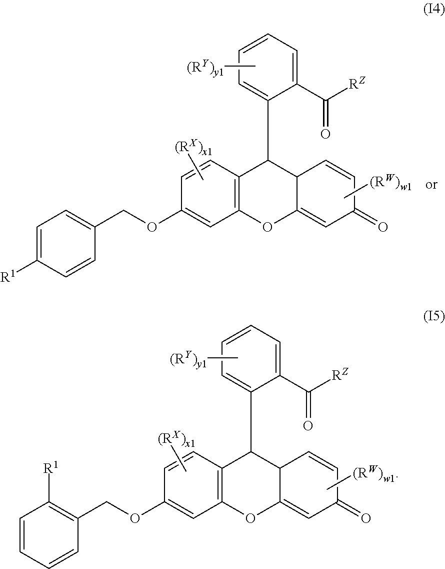

In embodiments, the compound of formula (I3) has the formula:

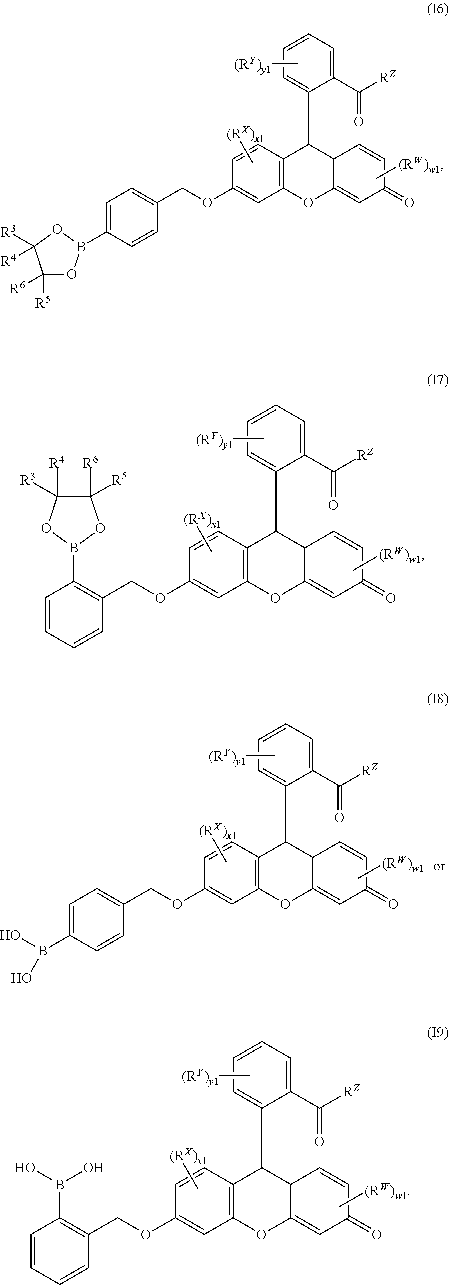

In embodiments, R³, R⁴, R⁵ and R⁶ are independently C₁-C₅ substituted or unsubstituted alkyl.

In embodiments, the compound of formula (I3) has the formula:

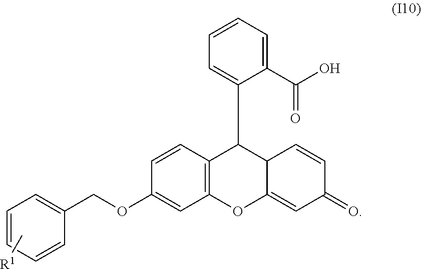

In embodiments, the compound of formula (I10) has the formula:

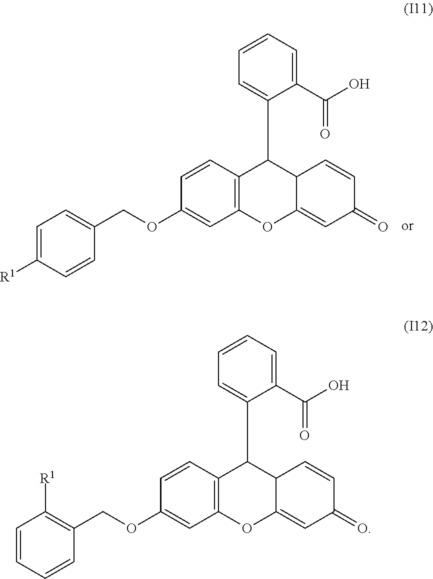

In embodiments, the compound of formula (I10) has the formula:

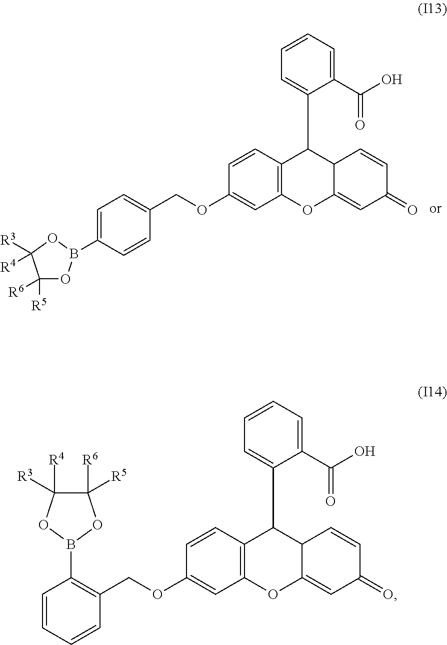

In embodiments, R³, R⁴, R⁵ and R⁶ are independently C₁-C₅ substituted or unsubstituted alkyl.

In embodiments, the compound of formula (I) has the formula:

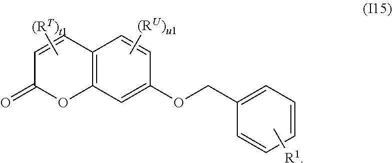

R¹, R^{T}, R^{U}, t1, and u1 are as described herein. In embodiments, R^{T} is hydrogen, halogen, —NH₂, —NO₂, —OH, —CN, —COOH, —CF₃, —OCH₃, —OCH₂CH₃, —CH₂Br, —CH₂COOH, substituted or unsubstituted alkyl, substituted or unsubstituted heteroalkyl, or substituted or unsubstituted aryl. In embodiments, R^{T} is formyl, methyl, ethyl, propyl, bromoacetal, acetyl, acetamide, methoxymethyl, allyloxy, propanoic acid, or succinimidyl ester. In embodiments, R^{U} is hydrogen, halogen, —OH, —NH₂, —CH₃, —NO₂, -EtNH₃, methoxy, acetyl, allyloxy, ethoxy, or acetic acid.

In embodiments, the compound of formula (I15) has the formula:

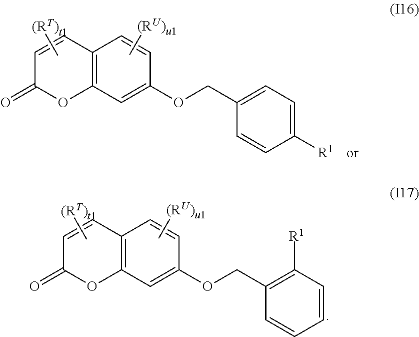

In embodiments, the compound of formula (I15) has the formula:

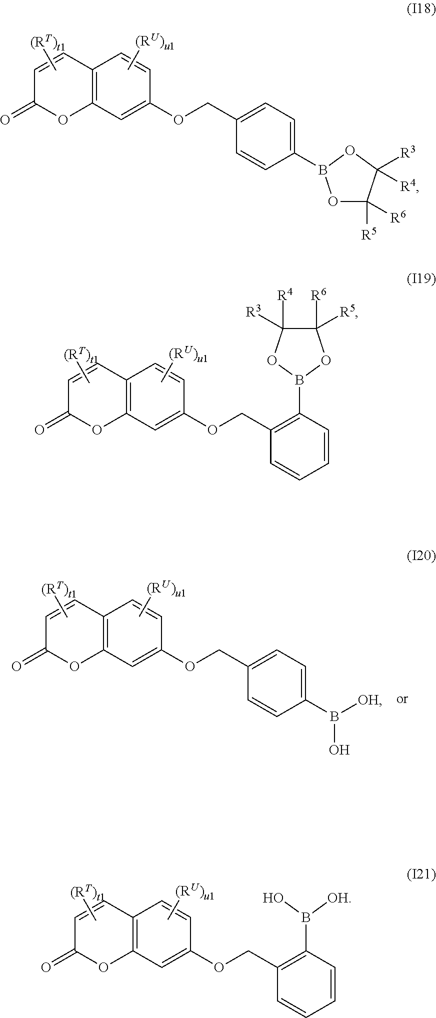

In embodiments, R³, R⁴, R⁵ and R⁶ are independently C₁-C₅ substituted or unsubstituted alkyl.

In embodiments, the compound of formula (I) has the formula:

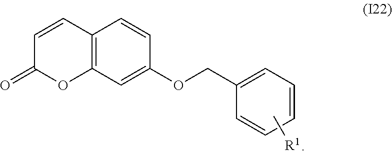

In embodiments, the compound of formula (I22) has the formula:

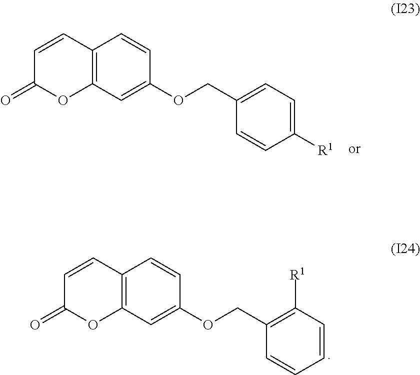

In embodiments, the compound of formula (I22) has the formula:

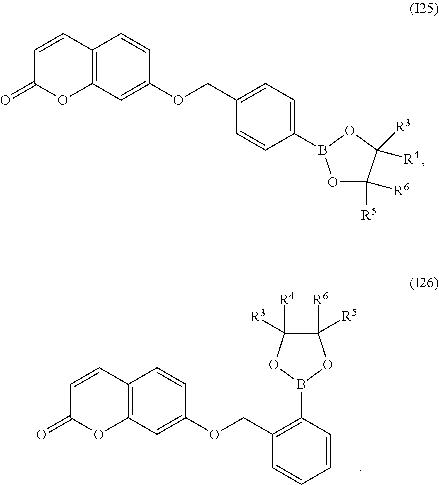

In embodiments, R³, R⁴, R⁵ and R⁶ are independently C₁-C₅ substituted or unsubstituted alkyl.

In embodiments, the compound of formula (I) has the formula:

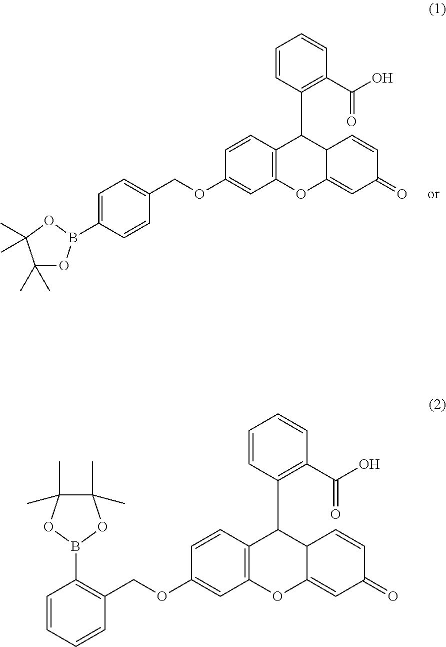 [SMILES: CC1(C)OB(c2ccc(COc3ccc4c(c3)OC3=CC(=O)C=CC3C4c3ccccc3C(=O)O)cc2)OC1(C)C ; CC1(C)OB(c2ccccc2COc2ccc3c(c2)OC2=CC(=O)C=CC2C3c2ccccc2C(=O)O)OC1(C)C]

(i.e. FBBE).

In embodiments, the compound of formula (I) has the formula:

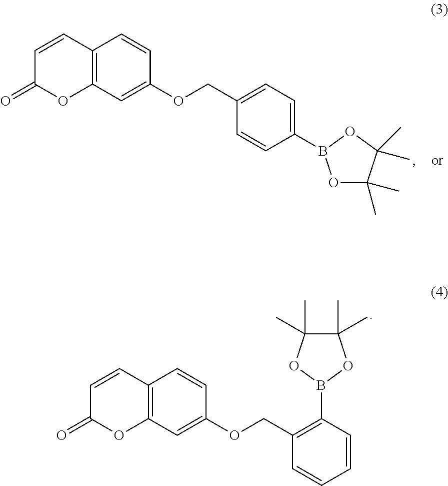 [SMILES: CC1(C)OB(c2ccc(COc3ccc4ccc(=O)oc4c3)cc2)OC1(C)C ; CC1(C)OB(c2ccccc2COc2ccc3ccc(=O)oc3c2)OC1(C)C]

(i.e. CBBE).

In embodiments, the compound of formula (I) has formula:

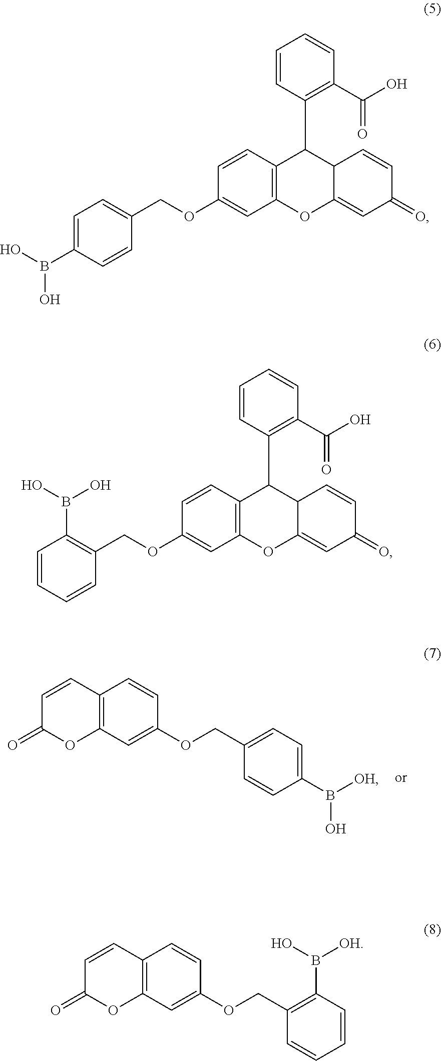 [SMILES: O=C1C=CC2C(=C1)Oc1cc(OCc3ccc(B(O)O)cc3)ccc1C2c1ccccc1C(=O)O ; O=C1C=CC2C(=C1)Oc1cc(OCc3ccccc3B(O)O)ccc1C2c1ccccc1C(=O)O ; O=c1ccc2ccc(OCc3ccc(B(O)O)cc3)cc2o1 ; "or" ; O=c1ccc2ccc(OCc3ccccc3B(O)O)cc2o1]

In embodiments, the compound of formula (II) has the formula:

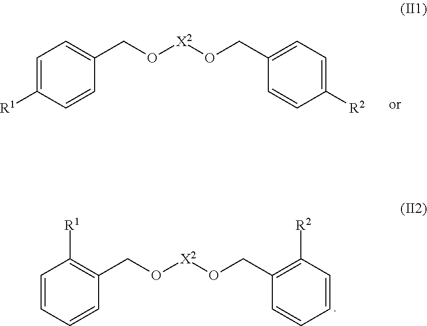

R¹, R², X¹, and X² are as described herein. In embodiments, R¹ is a compound of formula (V), where R³, R⁴, R⁵ and R⁶ are independently methyl. In embodiments, R² is a compound of formula (V), where R³, R⁴, R⁵ and R⁶ are independently methyl. In embodiments, R¹ and R² are identical. In embodiments, R¹ and R² are independently a compound of formula (V) or —B(OH)₂. In embodiments, R¹ and R² are —B(OH)₂. X¹ may be a divalent pro-fluorescein (including conjugates, derivatives, and analogues thereof) or a divalent pro-coumarin (including conjugates, derivatives, and analogues thereof).

In embodiments, the compound of formula (II) has the formula:

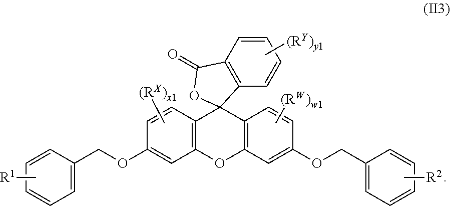

R¹, R², R^{W}, R^{X}, R^{Y}, R^{Z}, w1, x1, and y1 are as described herein. In embodiments, R^{W} and R^{X} are independently hydrogen, halogen or —OCH₃. In embodiments, R^{Y} and R^{Z} are independently hydrogen, halogen, —OH, —OCH₃, —COOH, —CH₂Br, Iodoacetamido, N-succinimidyl ester, hexanoic acid, hexanoic acid N-hydroxysuccinimide ester, or isothiocyanate.

In embodiments, the compound of formula (II3) has the formula:

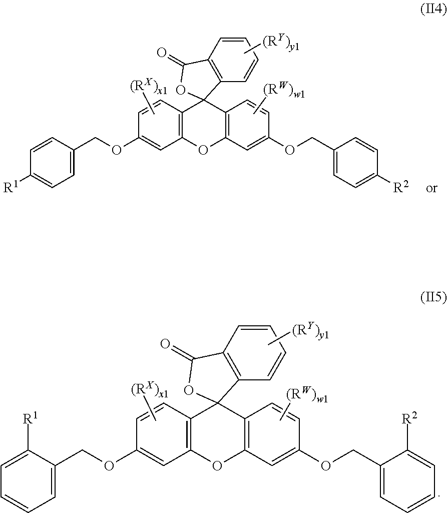

In embodiments, the compound of formula (II3) has the formula:

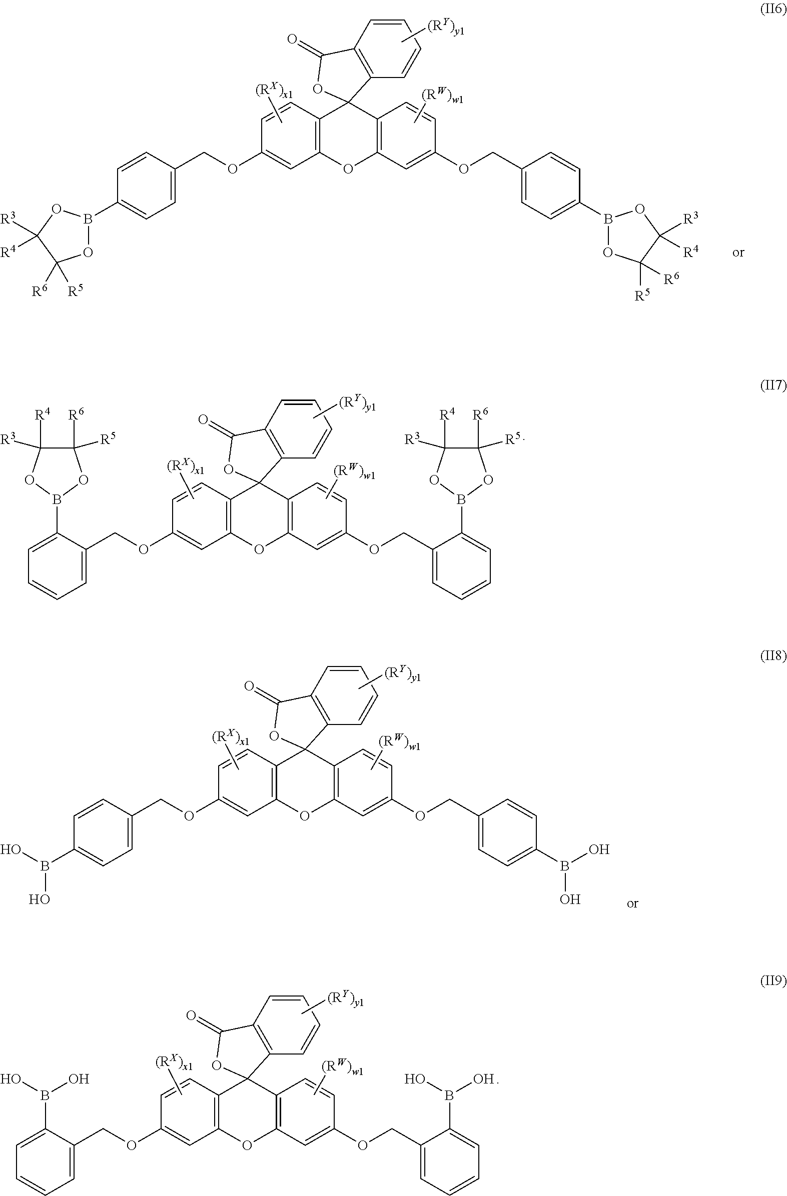

In embodiments, the compound of formula (II) has the formula:

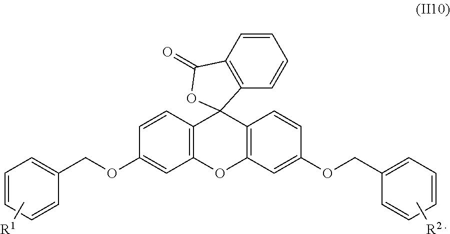

In embodiments, the compound of formula (II10) has the formula:

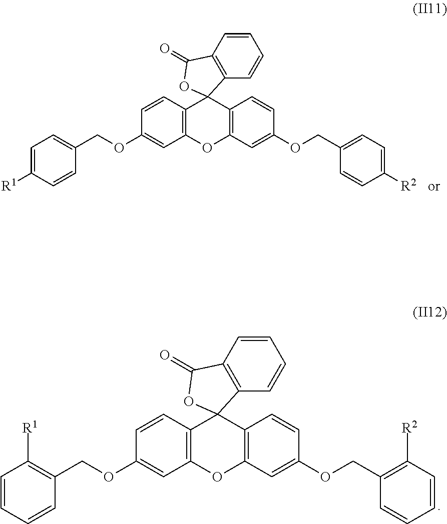

In embodiments, the compound of formula (II10) has the formula:

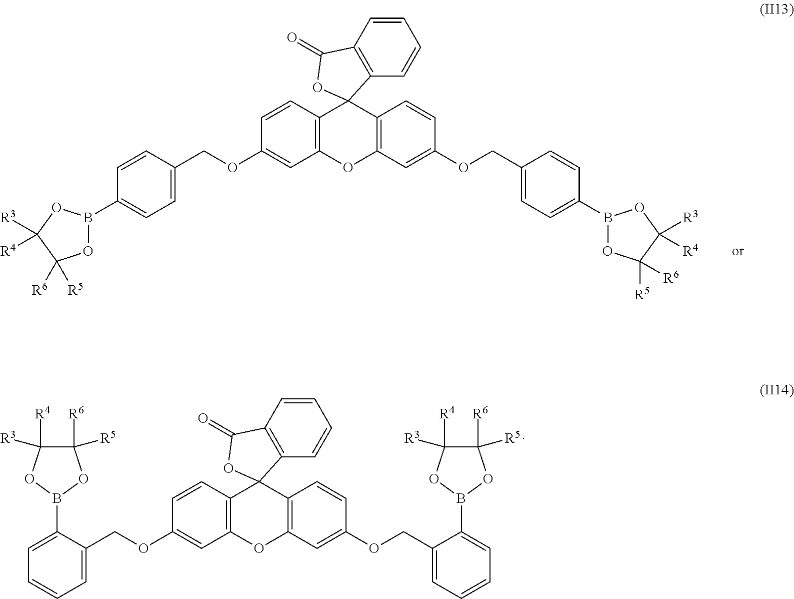

In embodiments, R³, R⁴, R⁵ and R⁶ are independently C₁-C₅ substituted or unsubstituted alkyl.

In embodiments, the compound of formula (II) has the formula:

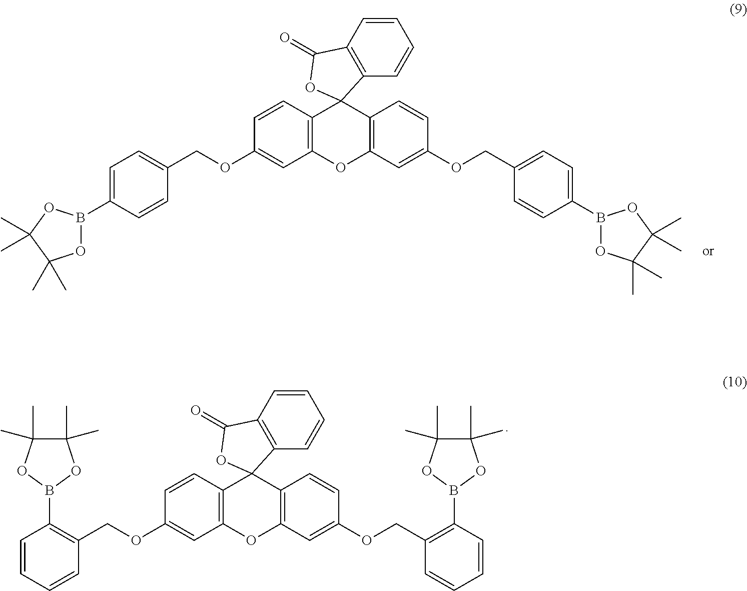

(i.e. FBBBE).

In embodiments, the compound of formula (II) has the formula:

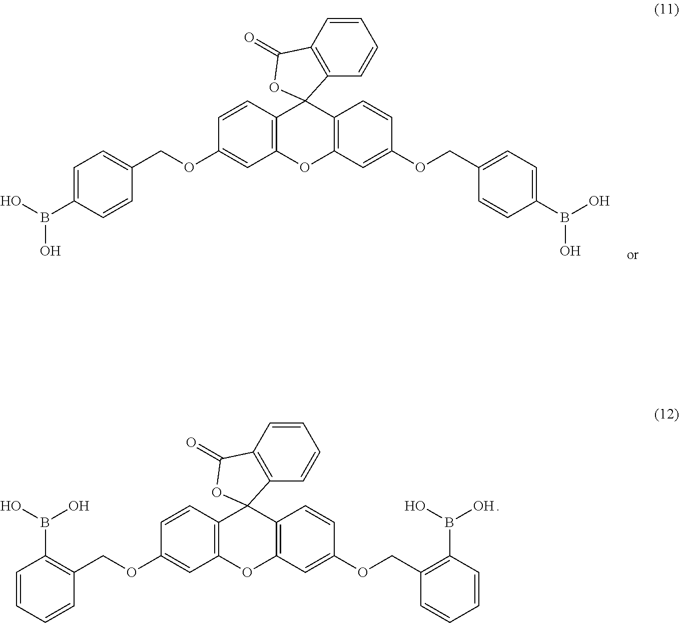 [SMILES: O=C1OC2(c3ccc(OCc4ccc(B(O)O)cc4)cc3Oc3cc(OCc4ccc(B(O)O)cc4)ccc32)c2ccccc21 ; O=C1OC2(c3ccc(OCc4ccccc4B(O)O)cc3Oc3cc(OCc4ccccc4B(O)O)ccc32)c2ccccc21]

Also provided herein are compounds having formula (III):

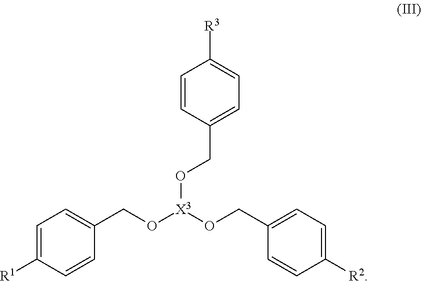

R¹ and R² are as described herein (e.g. formula (I) and (II), including embodiments thereof). R³ is —B(OH)₂ or a moiety of formula (V) as described herein, including embodiments thereof. In embodiments, R¹, R², and R³ are identical. X³ is a trivalent pro-fluorophore. In embodiments, X³ is trivalent pro-fluorescein, trivalent pro-coumarin, trivalent pro-dansyl, trivalent pro-bimane, trivalent pro-texas red, trivalent pro-PyMPO, trivalent pro-BODIPY, trivalent pro-eosin, trivalent pro-rhodamine, or trivalent pro-cyanine, trivalent nile red, trivalent xanthone, trivalent substituted xanthene, trivalent flazo orange, trivalent Snarf1, or trivalent resorufin, including conjugates, derivatives, and analogues thereof.

Also provided herein are compounds of having formula (IV):

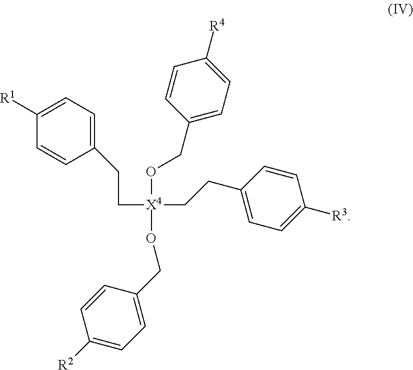

R¹, R², and R³, are as described herein (e.g. formula (I), (II) and (III)), including embodiments thereof. R⁴ is —B(OH)₂ or a moiety of formula (V) as described herein, including embodiments thereof. In embodiments, R¹, R², R³ and R⁴ are identical. X⁴ is a tetravalent pro-fluorophore. In embodiments, X⁴ is tetravalent pro-fluorescein, tetravalent pro-coumarin, tetravalent pro-dansyl, tetravalent pro-bimane, tetravalent pro-texas red, tetravalent pro-PyMPO, tetravalent pro-BODIPY, tetravalent pro-eosin, tetravalent pro-rhodamine, or tetravalent pro-cyanine, tetravalent nile red, tetravalent xanthone, tetravalent substituted xanthene, tetravalent flazo orange, tetravalent Snarf1, or tetravalent resorufin, including conjugates, derivatives, and analogues thereof.

The compounds described herein (e.g. compounds of formula (I), (II), (III), and (IV), including embodiments thereof) may be synthesized according to the scheme shown in FIG. 2.

The compounds described herein are useful in methods of detecting a ROS compound. Such methods include contacting a compound of formula (I) or formula (II) with a ROS compound. The ROS compound is allowed to react with the compound of formula (I) and remove R¹ or allowed to react with the compound of formula (II) and remove R¹ and R². The ROS compound may remove at least one of R¹ and R². Removing R¹ of the compound of formula (I) or R¹ and R² of the compound of formula (II) forms a fluorescent compound. In embodiments, removing at least one of R¹ and R² of the compound of formula (II) forms a fluorescent compound. The fluorescent compound is detected thus detecting the ROS compound. The detection may be performed using techniques known in the art. In embodiments, the subject is a cell.

In embodiments, the detecting includes quantifying an amount or level of the ROS compound. The quantifying may include determining an amount of fluorescence from the fluorescent compound. The amount of the fluorescence may be linearly proportional to the concentration of the ROS species (i.e. a greater concentration of ROS species results in a greater amount of fluorescence). In embodiments, the amount is compared to a control to determine the fold increase upon conversion of the pro-fluorophore to the fluorophore (e.g. fluorescent turn-on response).

The ROS compound may be H₂O₂, OCl⁻, or ONOO⁻. In embodiments, the ROS compound is H₂O₂. In embodiments, the ROS compound is OCL. In embodiments, the ROS compound is ONOO⁻. R¹ and X¹ of the compound of formula (I) are as described herein, including embodiments thereof. In embodiments, R¹ is a moiety of formula (V), where R³, R⁴, R⁵, and R⁶ are as described herein. In embodiments, R³, R⁴, R⁵, and R⁶ independently substituted or unsubstituted alkyl. R³, R⁴, R⁵, and R⁶ may independently be R⁷-substituted or unsubstituted alkyl, where R⁷ is as described herein, including embodiments thereof. R³, R⁴, R⁵, and R⁶ may independently be R⁷-substituted or unsubstituted C₁-C₅ alkyl. R³, R⁴, R⁵, and R⁶ may independently be methyl. In embodiments, R¹ is —B(OH)₂. In embodiments, X¹ is monovalent pro-fluorescein (including conjugates, derivatives and analogues thereof) or monovalent pro-coumarin (including conjugates, derivatives and analogues thereof). X¹ may be monovalent pro-fluorescein (including conjugates, derivatives and analogues thereof). X¹ may be monovalent pro-coumarin (including conjugates, derivatives and analogues thereof).

R¹, R², and X² of the compound of formula (II) are as described herein, including embodiments thereof. In embodiments, R¹ and R² are a moiety of formula (V), where R³, R⁴, R⁵, and R⁶ are as described herein. In embodiments, R¹ and R² are identical (e.g. R³, R⁴, R⁵, and R⁶ are identical). In embodiments, R¹ and R² are —B(OH)₂. In embodiments, R³, R⁴, R⁵, and R⁶ independently substituted or unsubstituted alkyl. R³, R⁴, R⁵, and R⁶ may independently be R⁷-substituted or unsubstituted alkyl, where R⁷ is as described herein, including embodiments thereof. R³, R⁴, R⁵, and R⁶ may independently be R⁷-substituted or unsubstituted C₁-C₅ alkyl. R³, R⁴, R⁵, and R⁶ may independently be methyl. In embodiments, X² is divalent pro-fluorescein (including conjugates, derivatives and analogues thereof). In embodiments, X² is divalent pro-fluorescein, divalent pro-rhodamine, divalent pro-nile red, divalent xanthone (e.g. substituted or unsubstituted), divalent xanthene (e.g. substituted or unsubstituted), divalent flazo orange, divalent Snarf1, or divalent resorufin, including conjugates, derivatives and analogues thereof.

The compounds herein are also useful in detecting a ROS compound in vivo. The method includes administering to a subject, a compound of formula (I) or formula (II). The compound of formula (I) is allowed to react with a ROS compound and remove R¹. The compound of formula (II) is allowed to react with a ROS compound and remove R¹ and R². The ROS compound may remove at least one of R¹ and R². Removing R¹ of the compound of formula (I) or R¹ and R² of the compound of formula (II) forms a fluorescent compound. In embodiments, removing at least one of R¹ and R² of the compound of formula (II) forms a fluorescent compound. The fluorescent compound is detected thus detecting the ROS compound in vivo. The detection may be performed using techniques known in the art.

R¹ and R² are as described herein, including embodiments thereof. In embodiments, R¹ and R² are a moiety of compound (V). R¹ and R² may be the identical. In embodiments, R¹ and R² are independently —B(OH)₂. X¹ and X² are as described herein, including embodiments thereof. In embodiments, X¹ is monovalent pro-fluorescein (including conjugates, derivatives and analogues thereof) or monovalent pro-coumarin (including conjugates, derivatives and analogues thereof). X¹ may be monovalent pro-fluorescein (including conjugates, derivatives and analogues thereof). X¹ may be monovalent pro-coumarin (including conjugates, derivatives and analogues thereof). In embodiments, X² is divalent pro-fluorescein (including conjugates, derivatives and analogues thereof). In embodiments, X² is divalent pro-fluorescein, divalent pro-rhodamine, divalent pro-nile red, divalent xanthone (e.g. substituted or unsubstituted), divalent xanthene (e.g. substituted or unsubstituted), divalent flazo orange, divalent Snarf1, or divalent resorufin, including conjugates, derivatives and analogues thereof.

In embodiments, R¹ and R² are removed at physiological levels of H₂O₂. In embodiments, R¹ and R² are removed at a H₂O₂ concentration of about 5 μM to about 200 μM. R¹ and R² may be removed at a H₂O₂ concentration of about 10 μM to about 200 μM. R¹ and R² may be removed at a H₂O₂ concentration of about 10 μM to about 100 μM. R¹ and R² may be removed at a H₂O₂ concentration of about 10 μM to about 90 μM. R¹ and R² may be removed at a H₂O₂ concentration of about 10 μM to about 80 μM. R¹ and R² may be removed at a H₂O₂ concentration of about 10 μM to about 70 μM. R¹ and R² may be removed at a H₂O₂ concentration of about 10 μM to about 60 μM. R¹ and R² may be removed at a H₂O₂ concentration of about 10 μM to about 50 μM. R¹ and R² may be removed at a H₂O₂ concentration of about 10 μM to about 40 μM. R¹ and R² may be removed at a H₂O₂ concentration of about 10 μM to about 30 μM. R¹ and R² may be removed at a H₂O₂ concentration of about 10 μM to about 20 μM.

R¹ and R² may be removed at a H₂O₂ concentration of about 5 μM to about 10 μM. R¹ and R² may be removed at a H₂O₂ concentration of about 5 μM to about 20 μM. R¹ and R² may be removed at a H₂O₂ concentration of about 5 μM to about 30 μM. R¹ and R² may be removed at a H₂O₂ concentration of about 5 μM to about 40 μM. R¹ and R² may be removed at a H₂O₂ concentration of about 5 μM to about 50 μM. R¹ and R² may be removed at a H₂O₂ concentration of about 5 μM to about 60 μM. R¹ and R² may be removed at a H₂O₂ concentration of about 5 μM to about 70 μM. R¹ and R² may be removed at a H₂O₂ concentration of about 5 μM to about 80 μM. R¹ and R² may be removed at a H₂O₂ concentration of about 5 μM to about 90 μM. R¹ and R² may be removed at a H₂O₂ concentration of about 5 μM to about 100 μM.

R¹ and R² may be removed at a H₂O₂ concentration of about 5, 10, 15, 20, 25, 30, 35, 40, 50, 60, 70, 80, 85, 90, 95, 100, 125, 150, 175, or about 200 μM. R¹ and R² may be removed at a H₂O₂ concentration of at least 5, 10, 15, 20, 25, 30, 35, 40, 50, 60, 70, 80, 85, 90, 95, 100, 125, 150, 175, or 200 μM. R¹ and R² may be removed at a H₂O₂ concentration of at least 5 μM. R¹ and R² may be removed at a H₂O₂ concentration of at least 10 μM. R¹ and R² may be removed at a H₂O₂ concentration of at least 20 μM. R¹ and R² may be removed at a H₂O₂ concentration of at least 30 μM. R¹ and R² may be removed at a H₂O₂ concentration of at least 40 μM. R¹ and R² may be removed at a H₂O₂ concentration of at least 50 μM. R¹ and R² may be removed at a H₂O₂ concentration of at least 60 μM. R¹ and R² may be removed at a H₂O₂ concentration of at least 70 μM. R¹ and R² may be removed at a H₂O₂ concentration of at least 80 μM. R¹ and R² may be removed at a H₂O₂ concentration of at least 90 μM. R¹ and R² may be removed at a H₂O₂ concentration of at least 100 μM. R¹ and R² may be removed at a H₂O₂ concentration of at least 150 μM. R¹ and R² may be removed at a H₂O₂ concentration of at least 200 μM.

R¹ and R² may be removed at a H₂O₂ concentration of at least 5 μM to at least 200 μM. R¹ and R² may be removed at a H₂O₂ concentration of at least 5 μM to at least 100 μM. R¹ and R² may be removed at a H₂O₂ concentration of at least 10 μM to at least 200 μM. R¹ and R² may be removed at a H₂O₂ concentration of at least 10 μM to at least 100 μM.

In embodiments, amount of fluorescence of the fluorescent compound (e.g. fluorescent turn-on response) is at least 5-, 10-, 20-, 30-, 40-, 50-, 55-, 60-, 65-, 70-, 80-, 90-, 95-, or 100-fold increased after contacting with the ROS compound. The amount of fluorescence of the fluorescent compound may be at least 10-fold increased after contacting with the ROS compound. The amount of fluorescence of the fluorescent compound may be at least 20-fold increased after contacting with the ROS compound. The amount of fluorescence of the fluorescent compound may be at least 30-fold increased after contacting with the ROS compound. The amount of fluorescence of the fluorescent compound may be at least 40-fold increased after contacting with the ROS compound. The amount of fluorescence of the fluorescent compound may be at least 50-fold increased after contacting with the ROS compound. The amount of fluorescence of the fluorescent compound may be at least 55-fold increased after contacting with the ROS compound. The amount of fluorescence of the fluorescent compound may be at least 60-fold increased after contacting with the ROS compound. The amount of fluorescence of the fluorescent compound may be at least 65-fold increased after contacting with the ROS compound. The amount of fluorescence of the fluorescent compound may be at least 70-fold increased after contacting with the ROS compound. The amount of fluorescence of the fluorescent compound may be at least 80-fold increased after contacting with the ROS compound. The amount of fluorescence of the fluorescent compound may be at least 90-fold increased after contacting with the ROS compound. The amount of fluorescence of the fluorescent compound may be at least 100-fold increased after contacting with the ROS compound.

The detecting of the ROS compound may include detecting the location of the ROS compound in the subject. Thus, in embodiments, detection of fluorescence at a location in the subject using the compounds herein indicates the location has ROS activity. In embodiments, the detection of the ROS compound may indicate a particular disease state or injury. In embodiments, the detecting includes quantifying a level or amount of the ROS compound in the subject. The amount of the fluorescence may be linearly proportional to the concentration of the ROS compound (i.e. a greater concentration of ROS compound results in a greater amount of fluorescence). The detection may be performed using techniques known in the art.

In embodiments, the monovalent pro-fluorophore moiety has substantially no fluorescence (e.g. less than 0.5%, 1%, 2%, 3%, 4%, 5%, 6%, 7%, 8%, 9%, 10%, 11%, 12%, 13%, 14%, 15%, 16%, 17%, 18%, 19%, or 20%) in the absence of a ROS compound when compared to the pro-fluorophore after contact with a ROS compound (e.g. fluorescent compound). In embodiments, the divalent pro-fluorophore moiety has substantially no fluorescence (e.g. less than 0.5%, 1%, 2%, 3%, 4%, 5%, 6%, 7%, 8%, 9%, 10%, 11%, 12%, 13%, 14%, 15%, 16%, 17%, 18%, 19%, or 20%) in the absence of a ROS compound when compared to the pro-fluorophore after contact with a ROS compound (e.g. fluorescent compound).

### I. EXAMPLES

### General Methods

All chemicals were purchased from commercial suppliers (Aldrich, Alfa Aesar, or Fisher) and used as provided. Chromatography was performed using a CombiFlash Rf-200 automated system from TeledyneISCO. ¹H and ¹³C NMR spectra were recorded on a Varian FT-NMR instrument running at 400 MHz at the Department of Chemistry and Biochemistry, University of California, San Diego. Mass spectrometry was performed at the Molecular Mass Spectrometry Facility in the Department of Chemistry and Biochemistry at the University of California, San Diego.

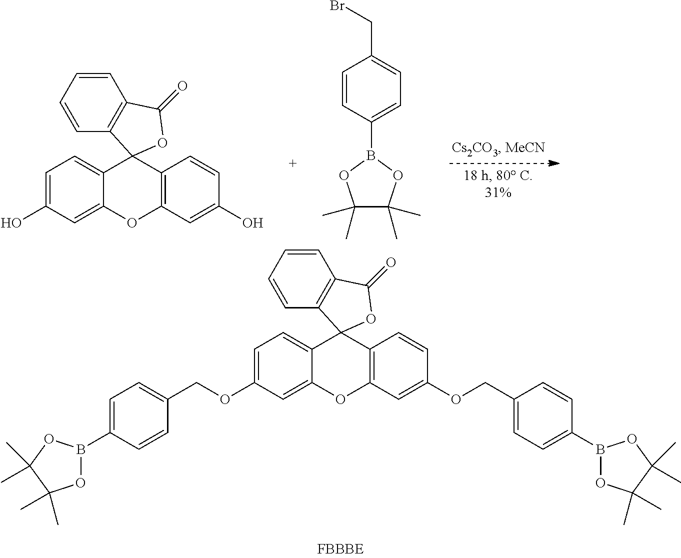

3′,6′-bis((4-(4,4,5,5-tetramethyl-1,3,2-dioxaborolan-2-yl)benzyl)oxy)-3H-spiro[isobenzofuran-1,9′-xanthen]-3-one (FBBBE): Fluorescein (2.45 g, 7.3 mmol) was dissolved in 50 mL MeCN. To this was added Cs₂CO₃ (7.21 g, 22.1 mmol) followed by 4-bromomethylphenyl boronic acid pinacol ester (6.57 g, 22.1 mmol). The reaction was held at reflux for 18 h under nitrogen. The mixture was then cooled to room temperature, filtered, and rinsed with CH₂Cl₂. The solvent was removed and the product was purified by silica gel chromatography, eluting with 20-30% EtOAc in hexanes to afford FBBBE in 31% yield (1.72 g, 2.3 mmol). ¹H NMR (400 MHz, CDCl₃): δ=8.01 (d, J=7.6 Hz, 1H), 7.85 (d, J=7.6 Hz, 4H), 7.62 (m, 2H), 7.43 (d, J=7.6 Hz, 4H), 7.15 (d, J=7.6 Hz, 1H), 6.82-6.81 (m, 2H), 6.70-6.67 (m, 4H), 5.11 (s, 4H), 1.35 (s, 24H). ¹³C NMR (100 MHz, CDCl₃): δ=169.7, 160.5, 153.4, 152.6, 139.6, 135.3, 135.2, 129.9, 129.4, 127.0, 126.7, 125.2, 124.2, 112.5, 111.7, 102.1, 84.1, 83.4, 70.4, 25.1. ESI-MS (+): m/z 765.44 [M+H]⁺, 787.37 [M+Na]⁺.

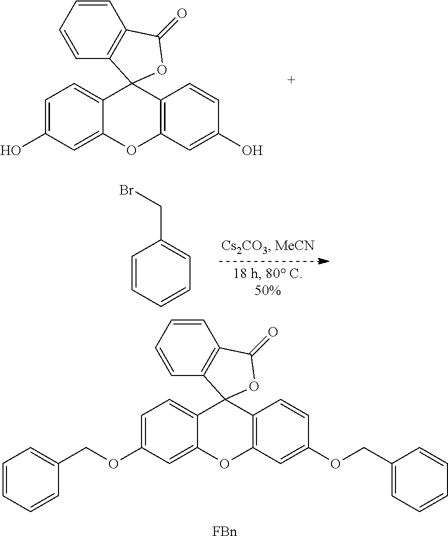

### 3′,6′-bis(benzyloxy)-3H-spiro[isobenzofuran-1,9′-xanthen]-3-one (FBn)

Fluorescein (0.75 g, 2.3 mmol) was dissolved in 15 mL MeCN. To this was added Cs₂CO₃ (2.21 g, 6.8 mmol) followed by benzyl bromide (1.1 mL, 9.0 mmol). The reaction was heated to reflux for 18 h under nitrogen. The mixture was then cooled to room temperature, filtered, and rinsed with CH₂Cl₂. The solvent was removed and the product was purified by silica gel chromatography, eluting with 5-25% EtOAc in hexanes to afford FBn in 50% yield (0.58 g, 1.1 mmol). ¹H NMR (400 MHz, CDCl₃): δ=8.03, (d, J=7.2 Hz, 1H), 7.69-7.60 (m, 2H), 7.45-7.33 (m, 10H), 7.17 (d, J=7.2 Hz, 1H), 6.85 (s, 2H), 6.71-6.79 (m, 4H), 5.10 (s, 2H). ¹³C NMR (100 MHz, CDCl₃): δ=169.7, 160.7, 153.3, 152.7, 136.5, 135.2, 129.9, 129.4, 128.9, 128.4, 127.7, 127.1, 125.2, 124.2, 112.5, 111.7, 102.1, 83.4, 70.5. ESI-MS (+): m/z 513.15 [M+H]⁺.

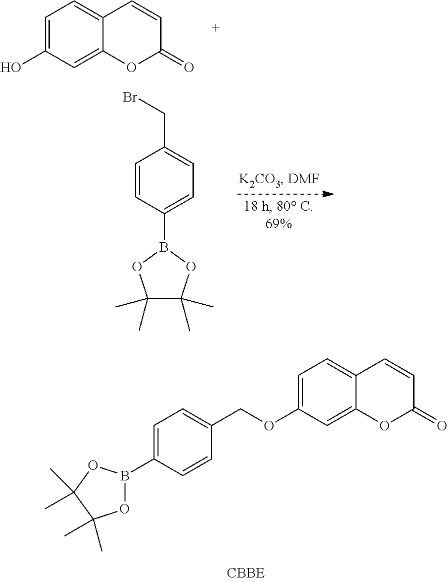

### 7-((4-(4,4,5,5-tetramethyl-1,3,2-dioxaborolan-2-yl)benzyl)oxy)-2H-chromen-2-one (CBBE)

7-hydroxycoumarin (1.25 g, 7.7 mmol) was dissolved in 15 mL of anhydrous DMF. To this was added of K₂CO₃ (3.20 g, 23.1 mmol) followed by 4-bromomethylphenyl boronic acid pinacol ester (6.18 g, 20.8 mmol). The reaction was heated to 80° C. under nitrogen for 18 h. The mixture was then cooled to room temperature, filtered, and rinsed with CH₂Cl₂. The filtrate was then washed with water and brine, dried over MgSO₄, filtered, and concentrated. The resulting residue was purified via silica gel chromatography eluting with 20% EtOAc in hexanes to afford the desired product in 69% yield (2.01 g, 5.3 mmol). ¹H NMR (400 MHz, CDCl₃): δ=7.83 (d, J=8.1 Hz, 2H), 7.63 (d, J=9.5 Hz, 1H), 7.42 (d, J=8.1 Hz, 2H), 7.37 (d, J=8.6 Hz, 1H), 6.90 (dd, J₁=8.6 Hz, J₂=2.4 Hz, 1H), 6.87 (d, J=2.4 Hz, 1H), 6.25 (d, J=9.5 Hz, 1H), 5.15 (s, 2H), 1.35 (s, 12H). ¹³C NMR (100 MHz, CDCl₃): δ=162.0, 161.4, 156.0, 143.61, 139.01, 135.4, 129.0, 126.8, 113.5, 113.4, 113.0, 102.2, 84.1, 70.6, 25.1. ESI-MS: (+) m/z: 378.95 [M+H]⁺, 395.79 [M+NH₄]⁺.

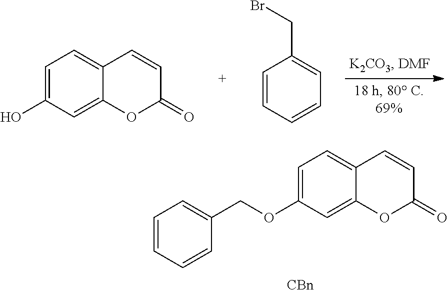

### 7-(benzyloxy)-2H-chromen-2-one (CBn)

7-Hydroxycoumarin (0.65 g, 4.0 mmol) was dissolved in 20 mL of MeCN. To this was added Cs₂CO₃ (2.61 g, 8.0 mmol) followed by benzyl bromide (1.0 mL, 8.0 mmol). The reaction was heated to 80° C. under nitrogen for 18 h.

The mixture was then cooled to room temperature, filtered, and rinsed with CH₂Cl₂. The solvent was removed and the resulting residue was purified by silica gel chromatography eluting with 20-50% EtOAc in hexanes to afford the purified product in 78% yield (0.78 g, 3.1 mmol). ¹H NMR (400 MHz, CDCl₃): δ=7.62 (d, J=9.6 Hz, 2H), 7.44-7.35 (m, 6H), 6.91 (dd, J₁=8.4 Hz, J₂=2.4 Hz, 1H), 6.87 (d, J=2.4 Hz, 1H), 5.12 (s, 2H). ¹³C NMR (100 MHz, CDCl₃): δ=162.1, 161.4, 156.0, 143.6, 136.0, 129.1, 129.0, 128.6, 127.8, 113.5, 113.4, 112.9, 102.1, 70.7. ESI-MS (+): m/z 253.03 [M+H]⁺, 269.72 [M+NH₄]⁺.

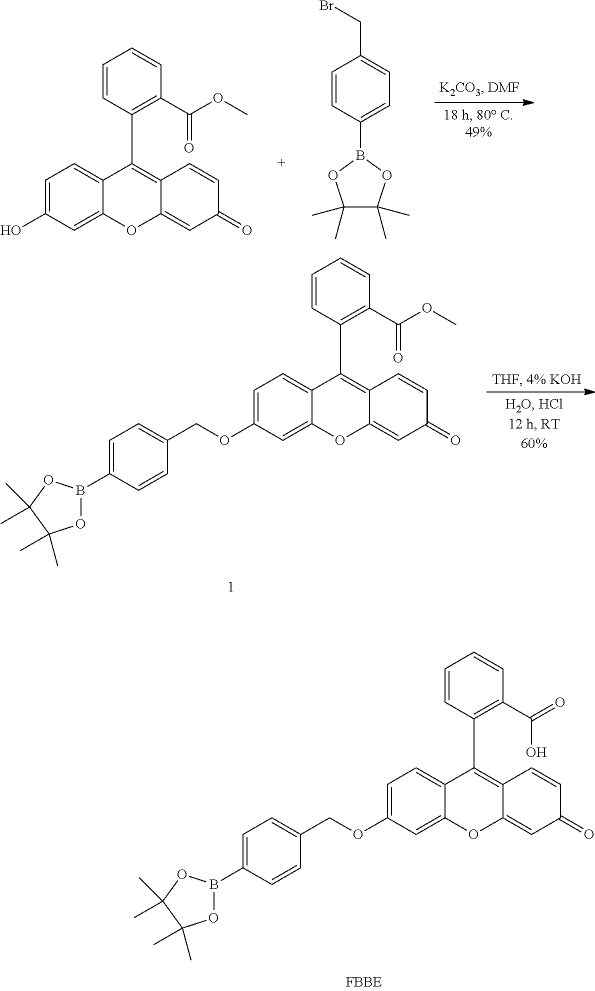

### Methyl 2-(3-oxo-6-((4-(4,4,5,5-tetramethyl-1,3,2-dioxaborolan-2-yl)benzyl)oxy)-3Hxanthen-9-yl)benzoate (1)

Fluorescein methyl ester (S. Y. Chen, et al., Bioconjugate Chem. 2010, 21, 979-987.) (2.09 g, 6.0 mmol) was dissolved in 20 mL DMF. To this was added potassium carbonate (2.50 g, 18.1 mmol) followed by 4-bromomethylphenyl boronic acid pinacol ester (3.23 g, 10.9 mmol). The reaction was held at 80° C. for 18 h under nitrogen. The mixture was then cooled to RT and filtered, rinsing with CH₂Cl₂. The solvent was removed and the resulting residue was purified by silica gel chromatography eluting 50-90% EtOAc in hexanes to afford 1 in 49% yield (1.66 g, 3.0 mmol). ¹H NMR (400 MHz, DMSO): δ=8.20 (d, J=8.0 Hz, 1H), 7.86 (td, J1=8.0 Hz, J2=1.2 Hz, 1H), 7.77 (td, J₁=8.0 Hz, J₂=1.2 Hz, 1H), 7.70 (d, J=8.0 Hz, 2H), 7.48 (t, J=8.0 Hz, 3H), 7.28 (d, J=2.8 Hz, 1H), 6.95 (dd, J₁=9.2 Hz, J₂=2.4 Hz, 1H), 6.83 (d, J=8.8 Hz, 1H), 6.78 (d, J=9.6 Hz, 1H), 6.37 (dd, J₁=9.6 Hz, J₂=2.0 Hz, 1H), 6.22 (d, J=2.0 Hz, 1H), 5.32 (s, 2H), 3.57 (s, 3H), 1.28 (s, 12H). ESI-MS (+): m/z 563.2 [M+H]⁺.

### 2-(3-oxo-6-((4-(4,4,5,5-tetramethyl-1,3,2-dioxaborolan-2-yl)benzyl)oxy)-3H-xanthen-9-yl)benzoic acid (FBBE)

1 (1.00 g, 1.8 mmol) was dissolved in 30 mL THF and 30 mL 4% KOH (aq). The reaction was held at RT for 12 h under nitrogen. The solvent was then removed and the resulting residue was brought up in 20 mL H₂O and 20 mL 1 M HCl. An extraction with EtOAc (4×50 mL) was performed, collecting the organic layer. This layer was dried over MgSO₄, filtered and concentrated. The solvent was removed and the resulting residue was purified by silica gel chromatography eluting 30-70% EtOAc in hexanes to afford FBBE in 56% yield (0.59 g, 1.1 mmol). ¹H NMR (400 MHz, CDCl₃): δ=8.01 (d, J=7.6 Hz, 1H), 7.83 (d, J=7.6 Hz, 1H), 7.68-7.59 (m, 2H), 7.42 (d, J=6.4 Hz, 2H), 7.16 (d, J=7.2 Hz, 1H), 6.80 (d, J=2.8 Hz, 1H), 6.73 (J=2.8 Hz, 1H), 6.67-6.65 (m, 2H), 6.58 (dd, J₁=8.8 Hz, J₂=0.8 Hz), 6.54-6.51 (m, 1H), 5.11 (s, 2H), 1.35 (s, 12H). ESI-MS (+): m/z 549.2 [M+H]⁺.

Luminescence Spectroscopy.

Fluorescence emission spectra were recorded with a Perkin-Elmer LS-55 fluorescence spectrometer. To a 2.0 mL solution of probe of varying concentrations in HEPES buffer (50 mM, pH 7.5) was added H2O2 in HEPES to monitor fluorescence turn-on. Spectra were monitored over time at room temperature with quartz cuvettes.

UV-Vis Spectroscopy.

Absorption spectra were taken on a Perkin-Elmer Lambda 25 UV-visible spectrophotometer. To a 1.0 mL solution of 50 μM probe in HEPES buffer (50 mM, pH 7.5) was added varying concentrations of H₂O₂ in HEPES to monitor deprotection. Spectra were monitored over time at room temperature with quartz cuvettes.

Calculation of Rate Constants.

The pseudo-first order rate constant was calculated by monitoring the absorption spectra over time in the presence of excess H₂O₂. To a 1.0 mL solution of 50 μM FBBBE or CBBE in HEPES buffer (50 mM, pH 7.5) was added H₂O₂ to a final concentration of 10 mM. Spectra were monitored over 5-15 min at room temperature with at least 100 spectra recorded. The change in absorption at 494 nm for FBBBE and 370 nm for CBBE were monitored. The rate constant (kobs) was determined by monitoring the appearance of the absorption peak by plotting the linear slope of ln [(Amax−A)/(Amax)] vs. time where Amax is the absorbance of a 50 μM sample of fluorescein (at 494 nm) for FBBBE and 7-hydroxycoumarin (at 370 nm) for CBBE. All rate constants were measured in triplicate using three independent experiments.

Calculation of Fluorescence Turn-On.

To quantitate the fluorescence turn-on, a sample of each probe (1 μM in 50 mM HEPES pH 7.5) was prepared. An excess of H₂O₂ (300 eq, 300 μM) was added to the latent fluorophore and spectra were recorded until no increase in fluorescence intensity was observed, signifying a complete reaction. The collected initial and final emission spectrum were integrated, and the ratio reports the fluorescence turn on.

Cell Imaging Experiments.

RAW 264.7 macrophages were grown in DMEM supplemented with 10% heat-inactivated fetal bovine serum, 2 mM L-glutamine, 100 μg/mL penicillin, and 100 μg/mL streptomycin (Life Technologies, Carlsbad, Calif.). For confocal microscopy, the RAW 264.7 cells were seeded at 250,000 in glass bottom 35 mm dishes (MatTek Corporation, Ashland, Mass.) and post-adhering (5 h), some dishes were treated with 1 μg/mL of LPS (Sigma-Aldrich, St. Louis, Mo.) for 24 h. Other dishes were treated the next day after plating with either 100 μM H₂O₂ for 1 h or PBS for 1 h. Cells were washed twice with PBS and fixed in ice-cold 95% ethanol for 15 min. Cells were twice rinsed with PBS and incubated with either 50 μM probe (FBBBE) or 50 μM control (FBn) for 1 h at room temperature. Cells were washed twice with PBS and coverslips were mounted over cells using Aqua Mount (Thermo Scientific, Waltham, Mass.). Confocal images were obtained at 80× magnification on an Olympus FV1000 confocal laser scanning microscope. Images were processed using ImageJ software (NIH).

Immunohistological Studies.

3-4 week old female C57BL/6J mice were purchased from Jackson Laboratory (Bar Harbor, Me.). Mice were sacrificed and perfused with sterile PBS. The brain and spinal cord were harvested and imbedded in Tissue-Tek® OCT Compound (Sakura Finetek USA, Torrance, Calif.) and frozen in dry ice before storage at −80° C. All animal studies were reviewed and approved by the University of California, San Diego Institutional Animal Care and Use Committee (La Jolla, Calif.).

For confocal microscopy, the frozen tissue was cut into 10-μm sections on a Leica cryomicrotome and mounted on slides. Tissue sections were fixed in ice-cold 95% ethanol for 15 min and rehydrated in PBS for 5 min at room temperature in a humidity chamber. Tissue sections were either incubated with PBS or 100 μM H₂O₂ for 1 h at room temperature in a humidity chamber, followed by a 1 h incubation with either 50 μM probe (FBBBE) or 50 μM control (FBn) and a 10 min nuclear stain (DAPI, 4′,6-diamidino-2-phenylindole). The sections were rinsed in PBS and hydrated in water. Coverslips were mounted using Aqua Mount (Thermo Scientific, Waltham, Mass.). Confocal images were obtained at 40× or 80× magnification on an Olympus FV1000 confocal laser scanning microscope. Images were processed using ImageJ software.

### Example 1

### Development of H₂O₂-Sensitive Probes

Fluorescein was selected as an inexpensive and widely used dye for cellular imaging due to its high quantum yield, low toxicity, and excellent water solubility.^{[24]}

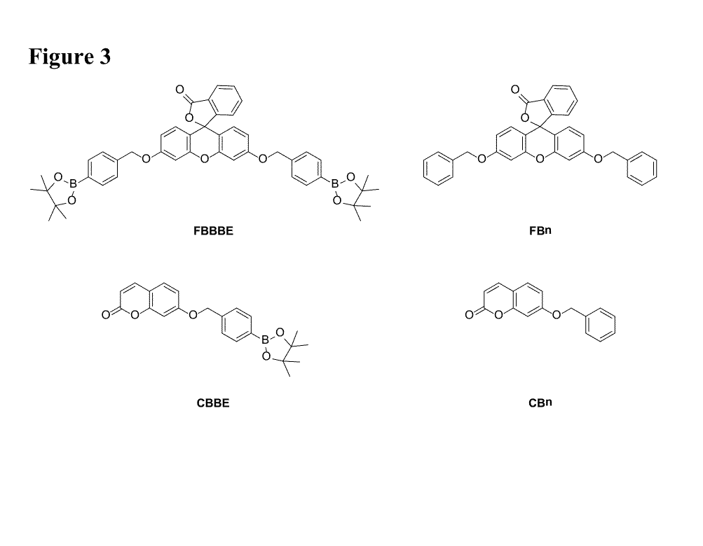

A protected fluorescein compound, fluorescein bis(benzyl boronic ester (FBBBE), was synthesized in one step from commercially available starting materials as shown in FIG. 2. In contrast, previously reported H₂O₂ probes employing boronic esters as triggers require 3-4 challenging and relatively low yielding synthetic steps.^{[14-15]} Additionally, the synthesis of FBBBE can be conducted on the gram scale (ESI), while most other reports describe quantities of probes in the low milligram range. FBBBE has pinacol boronic esters installed via a benzyl ether linkage to the fluorophore at both the 3″ and 6″ positions. The boronic ester moiety was chosen over the boronic acid in an attempt to enhance lipophilicity and, thus, cellular permeability. However, the boronic acid could be used in replacement of the boronic ester moiety. In order to evaluate the sensitivity of these probes to H₂O₂, a control compound was synthesized (FBn, FIG. 3).

### Example 2

### Exemplary Boronic Ester-Benzyl Ether Linked Compounds

To investigate the synthetic generality of this approach, a pro-fluorophore coumarin moiety (e.g. umbelliferone or 7-hydroxycoumarin) was protected with a boronic ester motif attached via a benzyl ether linkage (FIG. 3, CBBE). The corresponding control compound lacking the boronic ester trigger was also synthesized for evaluation (FIG. 3, CBn). Both CBBE and FBBBE were quite soluble in aqueous buffer, using only ˜1% DMSO (by volume) when preparing samples at micromolar concentrations.

### Example 3

### Evaluation of H₂O₂ Sensitivity

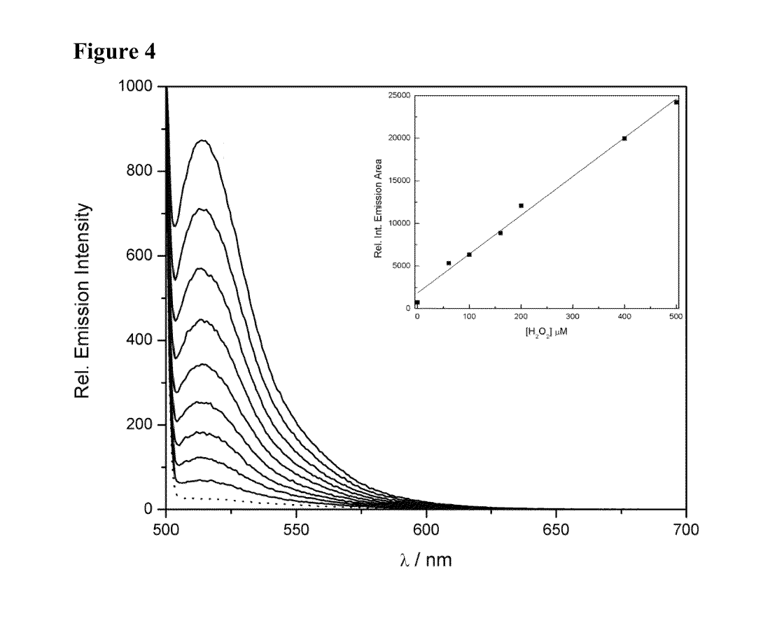

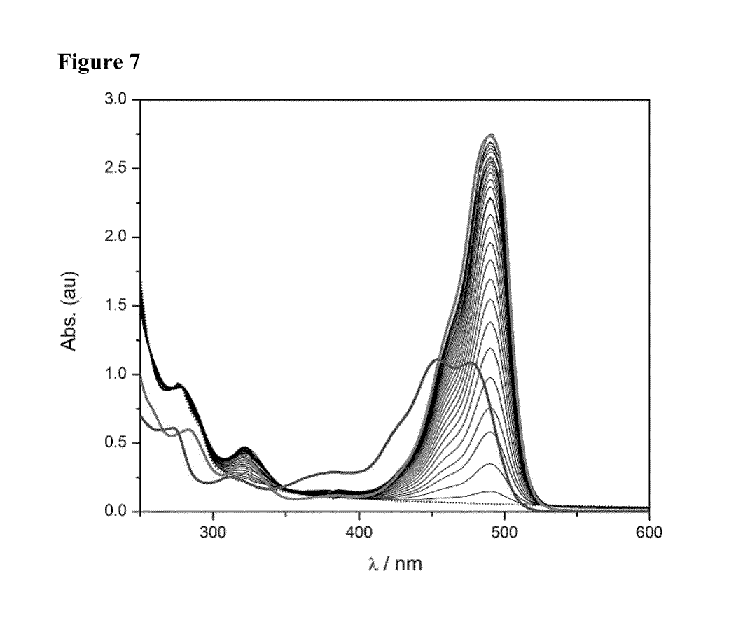

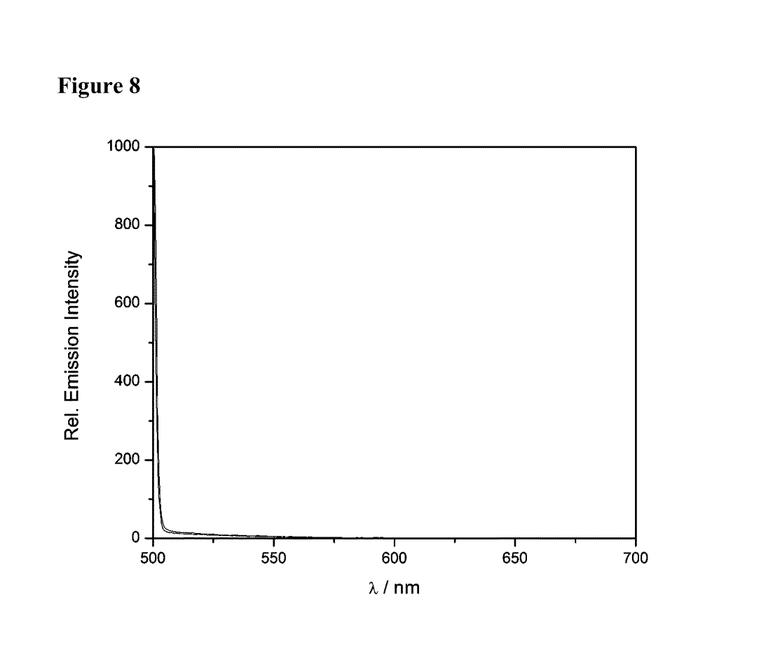

FBBBE and CBBE were evaluated for H₂O₂ sensitivity under simulated physiological conditions (50 mM HEPES, pH 7.5). For FBBBE, the installation of the benzyl protecting group forces the compound to remain in the closed lactone form, rendering FBBBE (and FBn) nonfluorescent. UV-Vis spectroscopy revealed a baseline absorption profile in the visible region for FBBBE (FIG. 7). Upon addition of H₂O₂ to FBBBE, a large increase in fluorescence was observed with a maximum λem=521 nm (FIG. 4). Along with the observed fluorescence increase, the absorption spectrum of FBBBE considerably changed upon H₂O₂ addition with a large increase in the visible absorption band (λmax=494 nm). The final spectrum recorded was identical to that of an authentic sample of fluorescein, providing additional proof that the probe was undergoing complete deprotection to the desired product (FIG. 7). The fluorescent response of FBBBE was characterized over a wide range of H₂O₂ concentrations to demonstrate a linear relationship in relative emission response (FIG. 4). FBBBE was incubated with different concentrations of H₂O₂ for 15 min and then the emission spectrum was collected. The spectra obtained were integrated between 500-700 nm and the relative emission area was plotted vs. H₂O₂ concentration, which showed a linear correlation (FIG. 4). As expected, control compound FBn does not show any changes in the emission or absorption spectra upon treatment with excess H₂O₂ (FIG. 8).

### Example 4

### Evaluation Cleavage Requirements

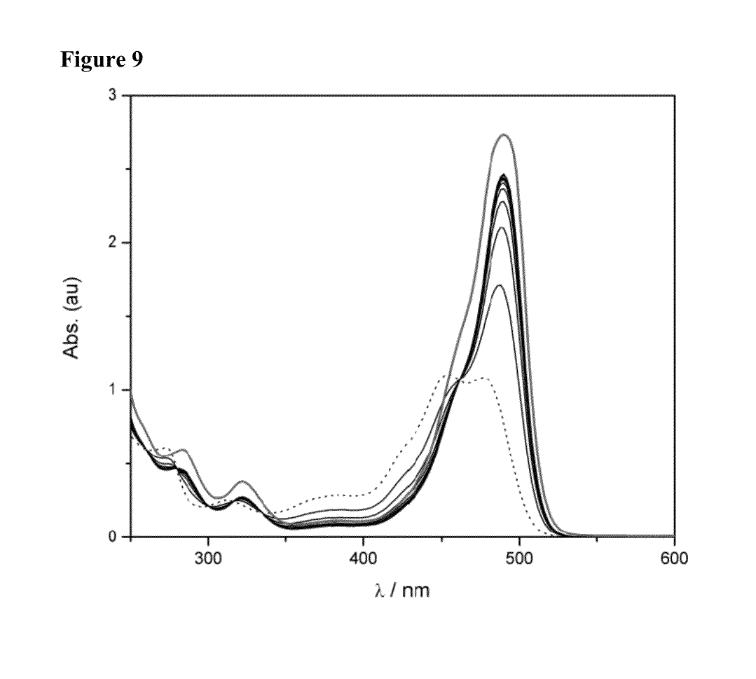

To determine whether the cleavage of one or both benzyl ether protecting groups from FBBBE is necessary to generate fluorescence, a second fluorescein derivative (in the free carboxylic acid form, FBBE), was synthesized that showed absorption in the visible region (FIG. 9). Emission spectroscopy revealed that this compound is highly fluorescent with an emission profile identical to that of fluorescein. After treating this probe with H₂O₂, the absorption spectra shifted to that of one matching fluorescein (FIG. 9). Taken together, these data suggest only one H₂O₂-mediated deprotection event was necessary to turn on fluorescence for FBBBE. However, as shown by absorption spectroscopy, treatment of FBBBE with H₂O₂ quickly converted the compound to fluorescein (FIG. 7), showing negligible accumulation of FBBE. This suggests that FBBE is a short-lived intermediate that does not contribute significantly to the sensor's response.

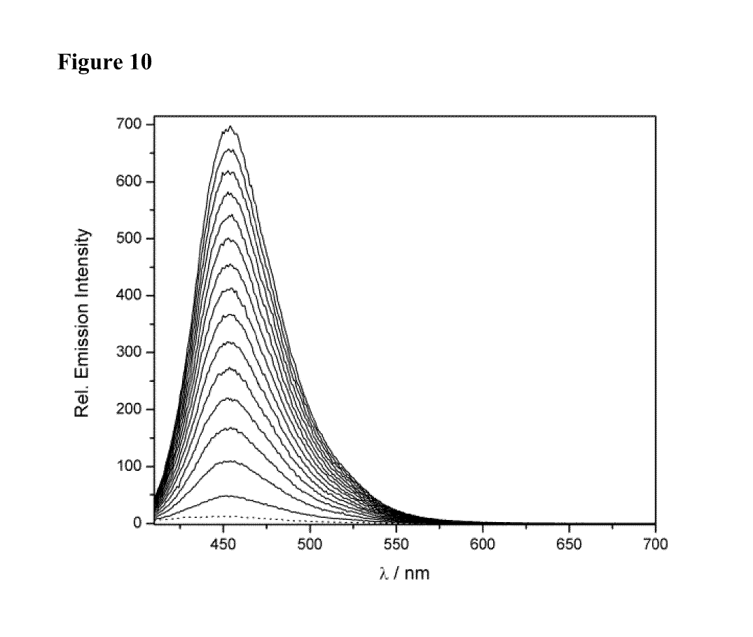

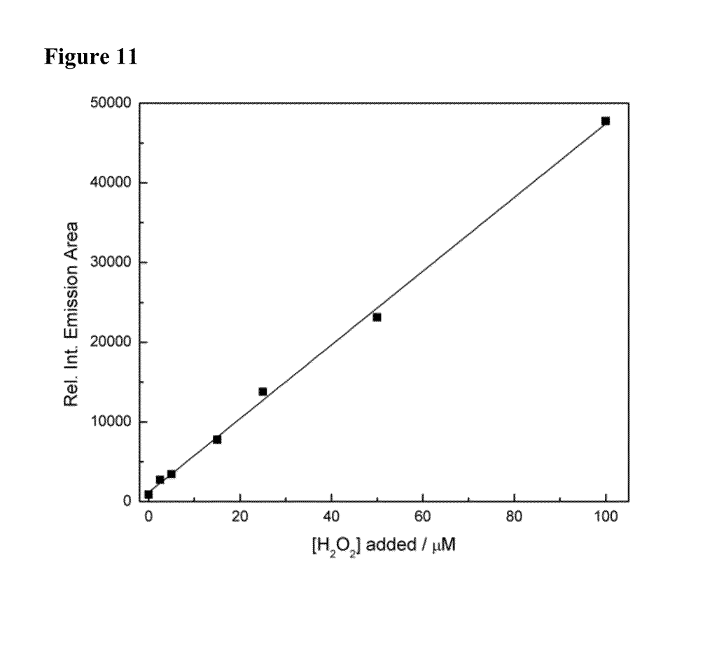

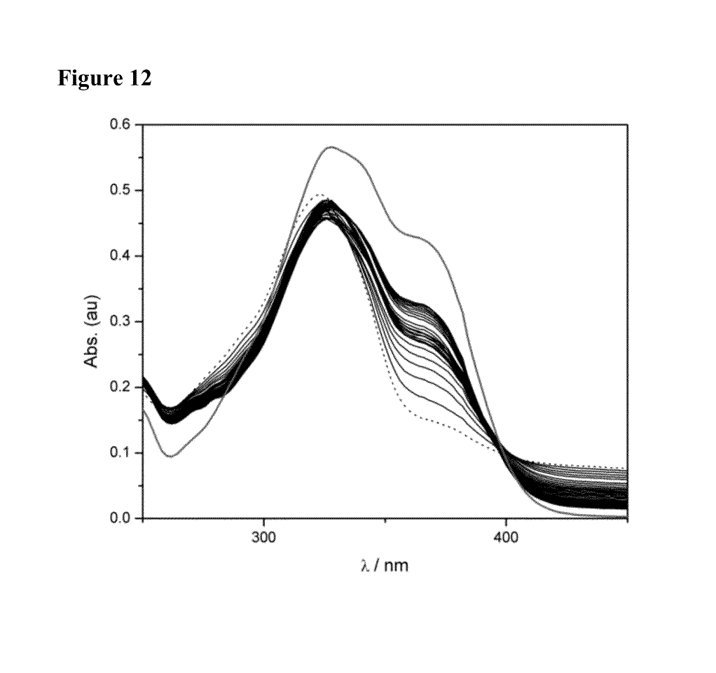

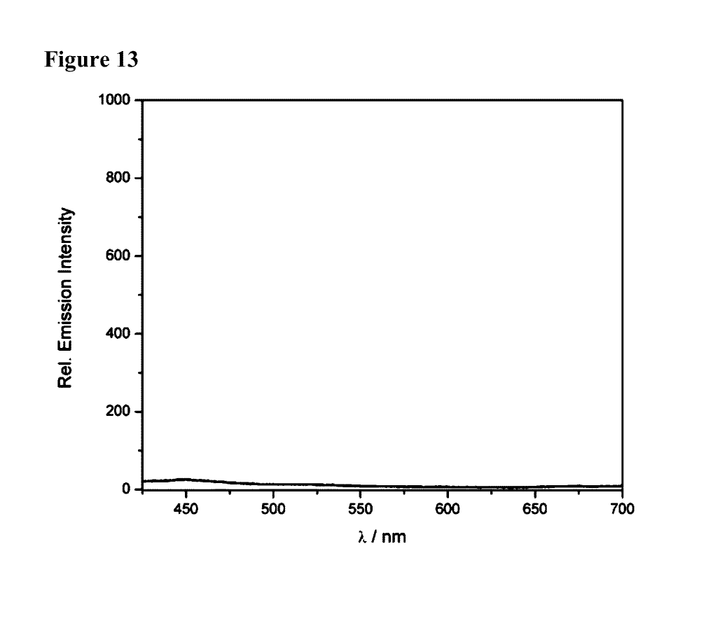

The coumarin-based compound, CBBE, was nonfluorescent in the absence of H₂O₂. The quenched fluorescence of CBBE quickly turned on in the presence of H₂O₂, with a maximum λem=453 nm (FIG. 10). A calibration plot similar to that of FBBBE was obtained for CBBE, showing a linear correlation between H₂O₂ added and observed fluorescent response (FIG. 11). Absorption spectroscopy also showed a shift in absorbance after H₂O₂ addition to CBBE, with the resulting spectrum matching an authentic sample of 7-hydroxycoumarin. (FIG. 12). The control compound, CBn, showed no change in the emission or absorption spectra after treatment with excess H₂O₂, as expected (FIG. 13).

### Example 5

### Further Sensitivity Studies

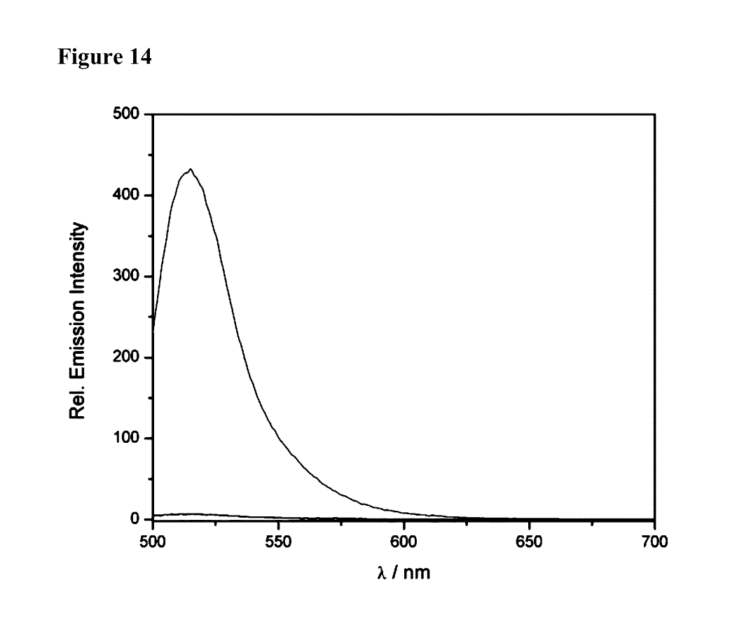

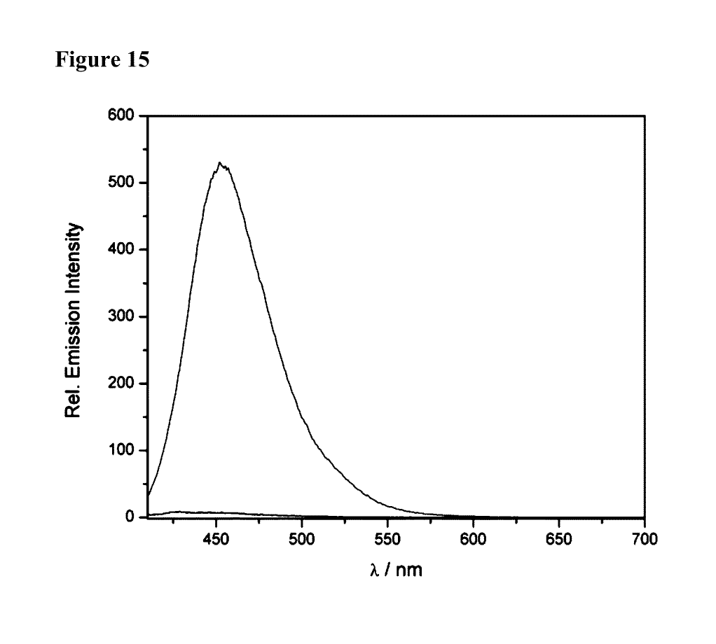

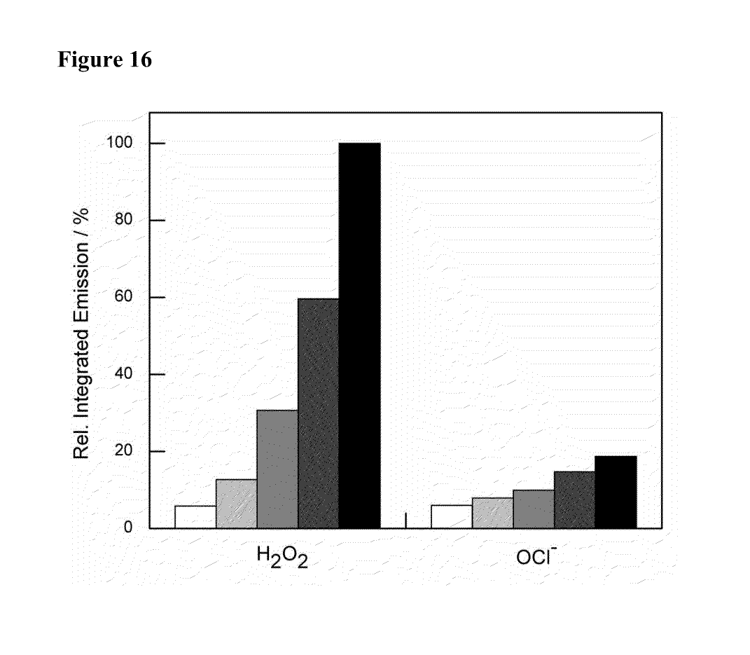

Additional studies were performed to quantify the fluorescence turn-on for each probe to directly compare the sensitivity to H₂O₂. FBBBE showed a 52-fold increase in fluorescence in response to excess H₂O₂, while CBBE showed a 57-fold increase (FIGS. 14 and 15). These fluorescent turn-on responses were excellent when compared with similar fluorophores reported for endogenous imaging of H₂O₂ (3-50 fold turn on),^{[12]} further demonstrating the value of these probes for sensing H₂O₂. To determine the sensitivity of these probes in a more quantitative fashion, pseudo first-order kinetic measurements were performed using UV-Vis absorption spectroscopy. The observed rate constants for FBBBE and CBBE activation by H₂O₂ were kobs=3.1×10-5 s-1 and kobs=4.6×10-3 s-1, respectively. It was determined that the deprotection of CBBE was much faster than FBBBE. This may be likely due to the fact that FBBBE contains two boronic ester triggers that are required for complete release of the fluorophore. Recently, it has been shown that boronic ester based probes can be sensitive to other biologically relevant ROS, such as hypochlorite (—OCl) and peroxynitrite (—ONOO).^{[26]} It was confirmed that FBBBE can also react with other biologically relevant ROS (e.g. —OCl), but is appears less sensitive to other ROS (e.g. —OCl) when compared to H₂O₂ (FIG. 16).

### Example 6

### Cellular Imaging Studies

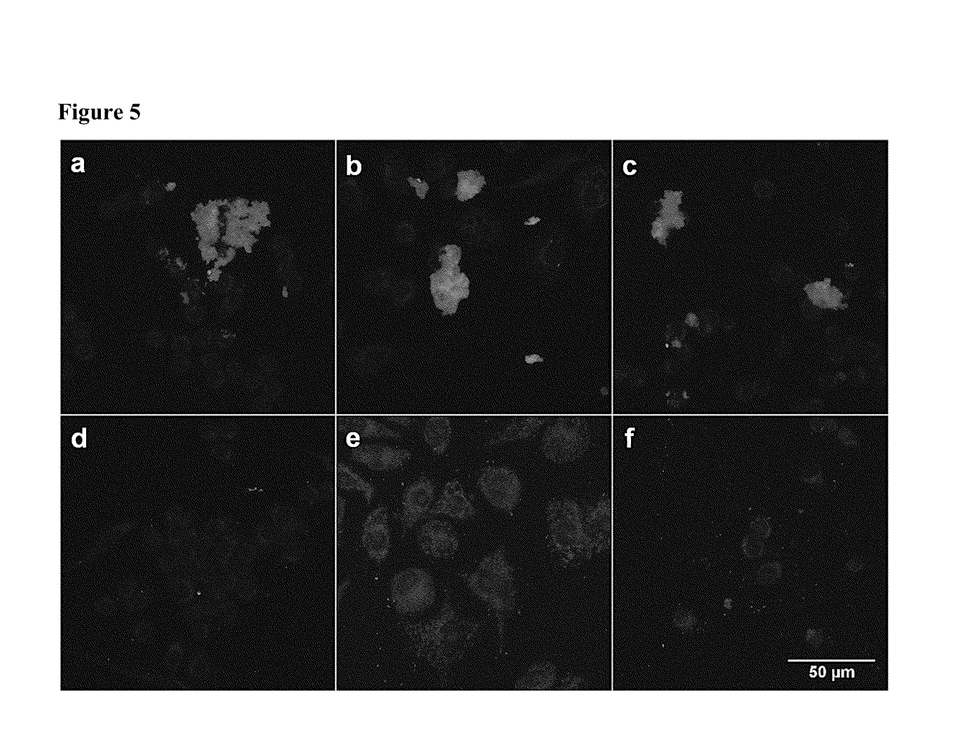

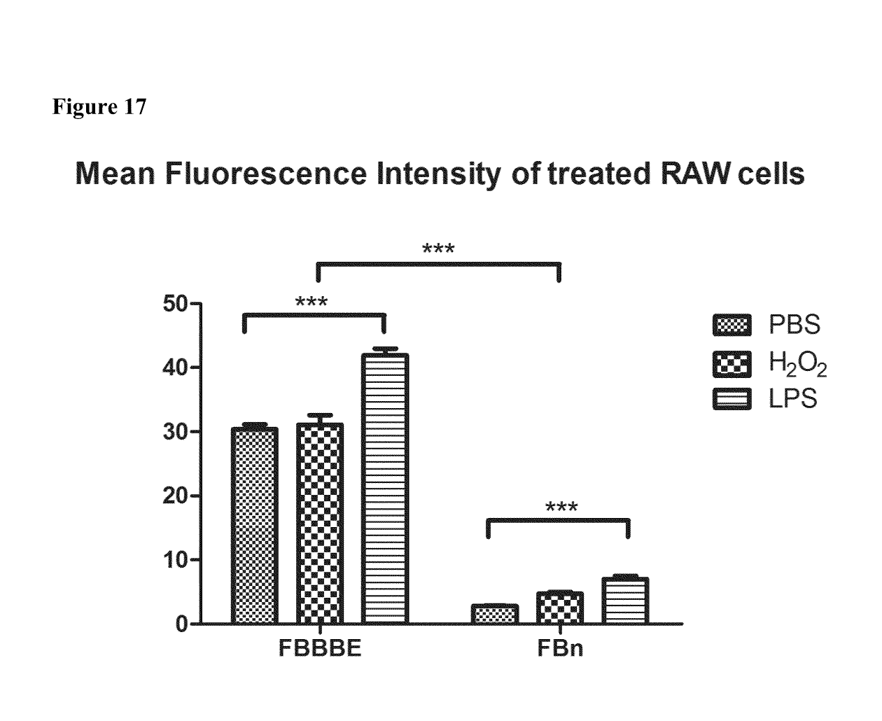

FBBBE was examined for H₂O₂ detection in biological samples via confocal microscopy. Murine macrophage RAW 264.7 cells were treated with PBS buffer (unstimulated cells), lipopolysaccharide (LPS, stimulated cells), or with exogenous H₂O₂, followed by incubation with either FBBBE (FIG. 5a-c) or FBn (FIG. 5d-f). The inflammatory effects of LPS in macrophages are well established and result in a burst of reactive oxygen species and reactive nitrogen intermediates among other inflammatory signatures.^{[27]} Fluorescence from activated FBBBE was observed in all cells (FIG. 5a-c). Quantification of the signal output revealed that mean fluorescence for cells treated with PBS or H₂O₂ (2 equiv) is not statistically different. However, the mean fluorescence of PBS or H₂O₂-treated cells was both statistically different (p<0.001) than those treated with LPS (FIG. 17). The observed results indicate that FBBBE is responsive to intracellular, endogenous levels of H₂O₂. The cells are washed and fixed prior to incubating with the probe. Experiments using the control probe FBn showed no turn on of fluorescence in any of the cells (FIG. 5d-f). Statistical analysis also showed a significant difference between the mean fluorescence signal output between FBBBE and FBn, as expected (FIG. 17).

### Example 7

### Tissue Imaging Experiments

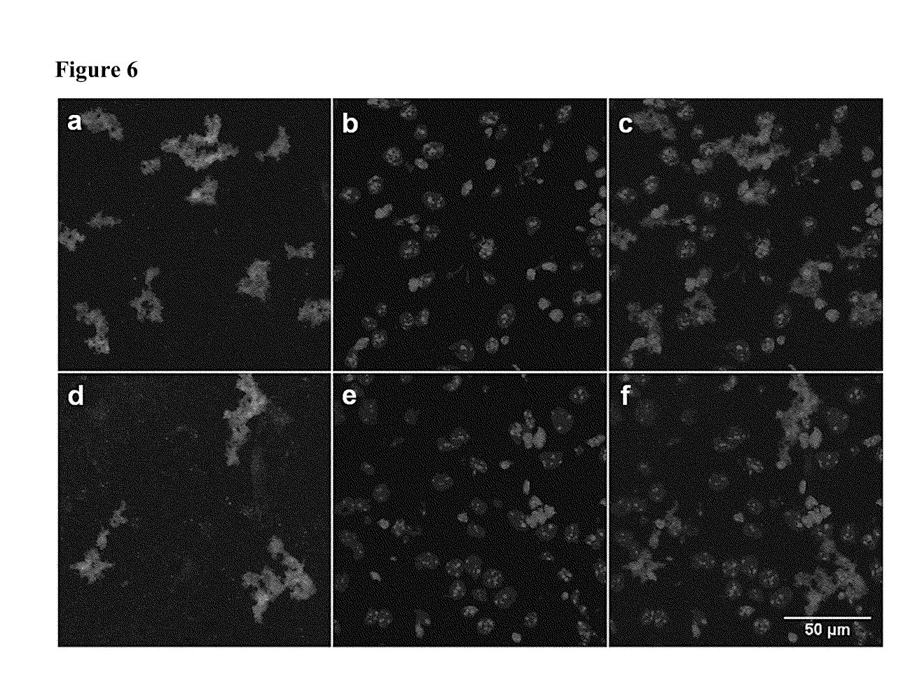

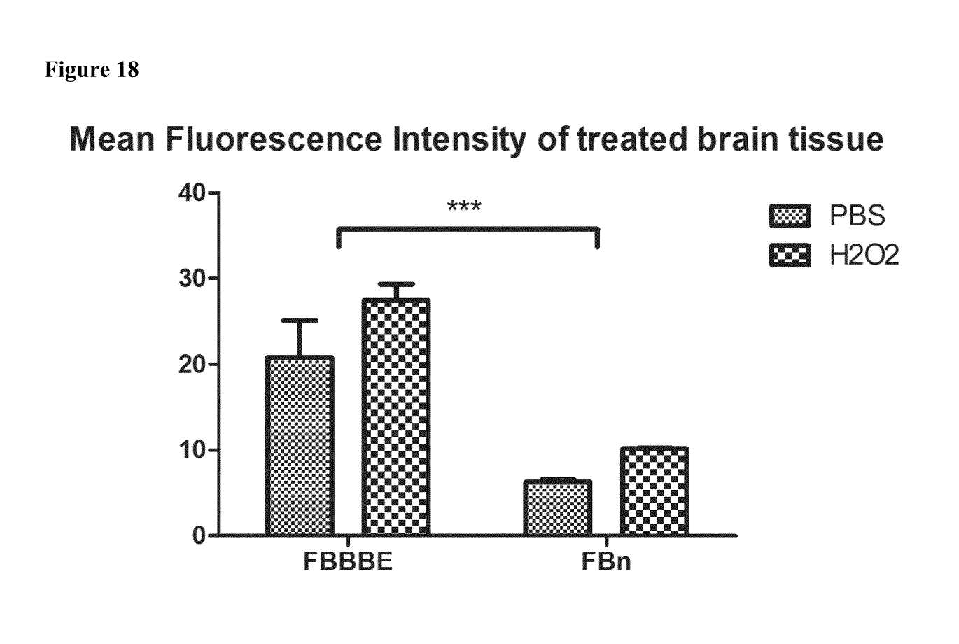

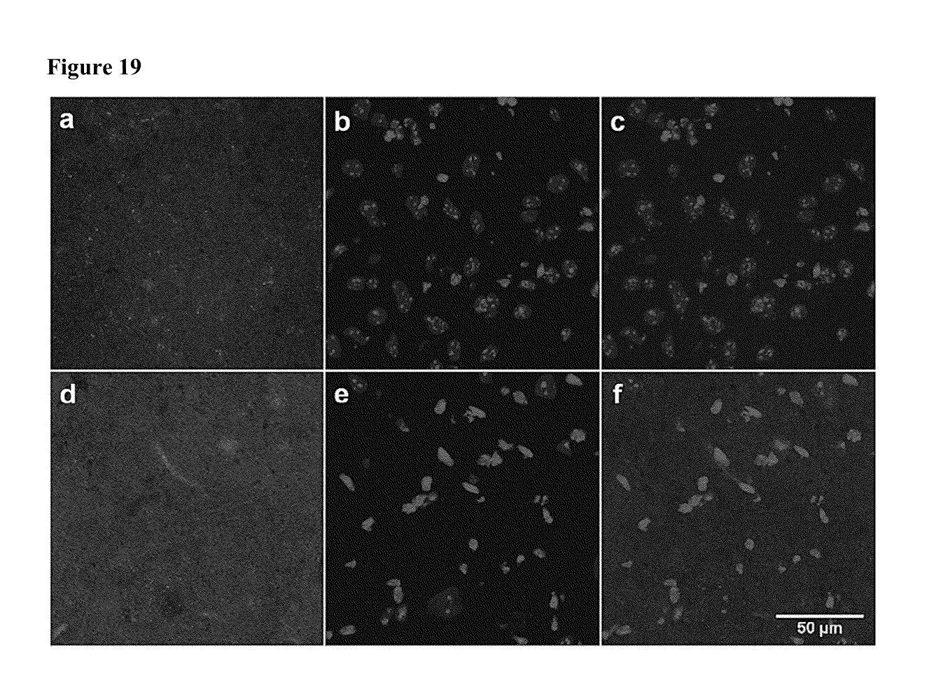

Cellular studies were extended to a more physiologically relevant model for H₂O₂ detection. H₂O₂ is particularly active in the brain and neuronal tissue, triggered by the metabolism of neurotransmitters.^{[28]} The brain and spinal cord from 3-4 week old female C57BL/6J mice were excised, frozen in OCT medium, and 10 μm sagittal cryosections were prepared for staining with FBBBE and FBn. At this age, the mice undergo extensive neuronal development and remodeling. Such activity has been associated with higher levels of neurotransmitter activity and ROS in both neurons and microglial cells in the brain. Consistent with the previous experiments, the sections were either incubated with PBS (unstimulated) or H₂O₂ (FIG. 6). Similar to the cell studies, endogenous levels of H₂O₂ were sufficient to activate the probe (FBBBE), and the exogenous addition of H₂O₂ did not alter the fluorescence levels observed (FIG. 6a, d). The probe appears to be activated intracellularly, based on its proximity to a nuclear stain (ESI, FIG. 6c, f). Quantitative analysis of FBBBE in the brain tissue treatments revealed that the mean fluorescence for tissues treated with PBS and those treated with H₂O₂ was not statistically different, consistent with the cellular studies (FIG. 18). When identical studies using the control FBn were performed, no fluorescence turn on was observed (FIG. 19). Additionally, control studies in the absence of the probe showed no fluorescence.

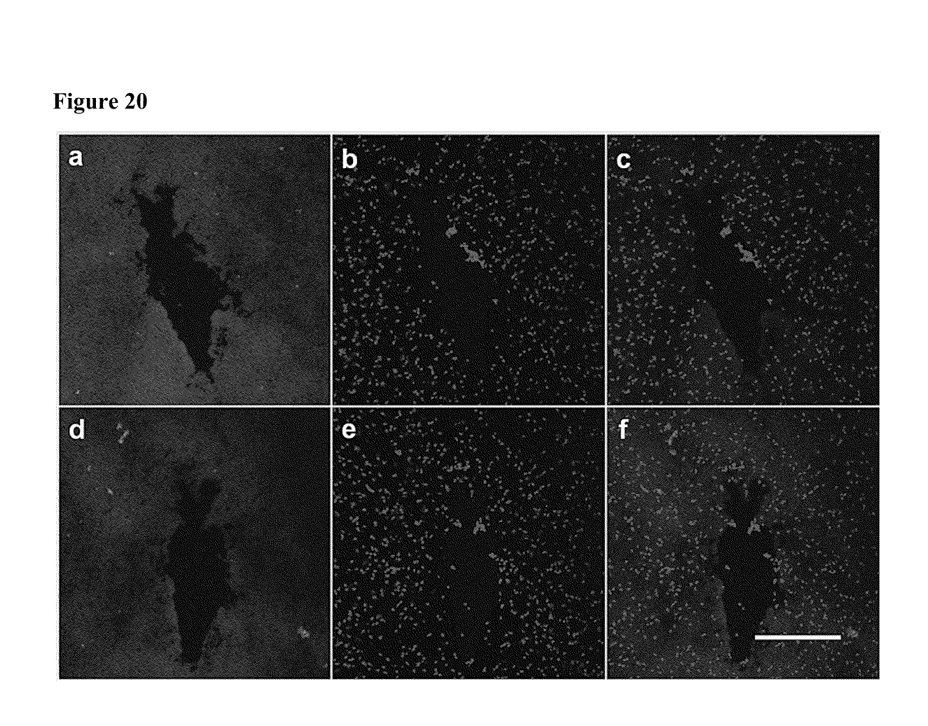

To further investigate whether FBBBE is activated by truly endogenous levels of H₂O₂, the probe was evaluated in the spinal cord. It is well established that the spinal cord consists primarily of white matter (myelinated axons), gray matter (axons), glial cells and some fibrous tissue with few axon terminals and activated macrophages. The presence of ROS is ubiquitous at axon terminals associated with neurotransmitter activity and metabolism.^{[28]} Thus, in a healthy, uninjured spinal cord, the presence of ROS such as H₂O₂ is not typical.^{[27c]} Spinal cord sections were either incubated with PBS (unstimulated) or H₂O₂, in a manner identical to the brain tissue studies. Confocal fluorescence images of the unstimulated spinal cord tissue treated with FBBBE showed that the probe remained essentially inactive with a weak fluorescence signal observed. The weak signal is attributable to tissue autofluorescence. Thus, the probe remained latent in areas of low ROS levels. The addition of exogenous H₂O₂ to the spinal cord tissue showed a minimal amount of fluorescence when compared to the brain tissue (FIG. 20). The lack of fluorescence signal from the H₂O₂-treated samples may be due to an inability of FBBBE to penetrate the spinal cord tissues, which is then subsequently washed away before the image is taken. Alternatively, FBBBE may be getting into the spinal cord tissues, but the externally added H₂O₂ is impermeable to the spinal cord cells and hence unable to activate FBBBE. In both tissues it appears that the exogenous addition of H₂O₂ does not potentiate the fluorescence signal, suggesting probe activation is caused by endogenous H₂O₂ activity in the brain not present in the spinal cord. These experiments suggest that the turn-on response of FBBBE is not found indiscriminately in all tissues types.

### CONCLUSION

Herein are reported the synthesis, reactivity, and imaging properties of a new class of fluorescence-based molecular probes for detecting endogenous H₂O₂ in biological systems. By appending a H₂O₂-sensitive boronic ester to a fluorophore moiety via a benzyl ether linkage, these fluorescent probes can be accessed in a single synthetic step using two commercially available starting materials. It was discovered, inter alia, under simulated physiological conditions, that both fluorescein and coumarin-based probes are sensitive for the detection of H₂O₂ in the biologically relevant micromolar concentration range. The fluorescein-based probe was particularly effective in imaging endogenous H₂O₂ levels with and without stimulation in murine macrophage cells using confocal miscroscopy and ex vivo imaging of endogenous H₂O₂ was effectively demonstrated in mouse brain. Overall, the combined synthetic ease, stability, solubility, fast reaction kinetics, and relatively high fluorescent turn-on response of these probes in comparison to other current fluorophores illustrate that the compounds herein represent accessible tools for molecular imaging for a wide range of researchers interested in the chemistry and biology of H₂O₂.

### II. REFERENCES

[1] M. Giorgio, M. Trinei, E. Migliaccio, P. G. Pelicci, Nat. Rev. Mol. Cell. Biol. 2007, 8, 722-728.
[2] a) S. G. Rhee, Science 2006, 312, 1882-1883; b) J. R. Stone, S. Yang, Antioxid. Redox Signaling 2006, 8, 243-270; c) B. D'AutrEaux, M. B. Toledano, Nat. Rev. Mol. Cell. Biol. 2007, 4, 278; d) C. C. Winterbourne, Nat. Chem. Biol. 2008, 4; eB. C. Dickinson, C. J. Chang, Nat. Chem. Biol. 2011, 7, 504-511.
[3] K. B. Beckman, B. N. Ames, Phys. Rev. 1998, 78, 547-581.
[4] a) J. P. Fruehauf, F. L. Meyskens, Clin. Cancer Res. 2007, 13, 789-794; b) T. Finkel, M. Serrano, M. A. Blasco, Nature 2007, 448, 767-774; c) K. Ishikawa, Science 2008, 321, 342-342.
[5] K. Sugamura, Free Radical Biol. Med. 2011, 51, 978-992.
[6] K. J. Barnham, C. L. Masters, A. I. Bush, Nat. Rev. Drug Disc. 2004, 3, 205-214.
[7] N. Houstis, E. D. Rosen, E. S. Lander, Nature 2006, 440, 944-948; D. Jay, H. Hitomi, K. Griendling, Free Radical Biol. Med. 2006, 40.
[8] M. H. Lim, S. J. Lippard, Acc. Chem. Res. 2007, 40, 41-51; bL. E. McQuade, S. J. Lippard, Curr. Opin. Chem. Biol. 2010, 14, 43-49.
[9] a) D. Yang, H. L. Wang, Z. N. Sun, N. W. Chung, J. G. Shen, J. Am. Chem. Soc. 2006, 128, 6004-6005; b) Z. N. Sun, H. L. Wang, F. Q. Liu, Y. Chen, P. K. H. Tam, D. Yang, Org. Lett. 2009, 11, 1887-1890.
[10] a) H. Maeda, K. Yamamoto, Y. Nomura, I. Kohno, L. Hafsi, N. Ueda, S. Yoshida, M. Fukuda, Y. Fukuyasu, Y. Yamauchi, N. Itoh, J. Am. Chem. Soc. 2005, 127, 68-69; b) K. M. Robinson, M. S. Janes, M. Pehar, J. S. Monette, M. F. Ross, T. M. Hagen, M. P. Murphy, J. S. Beckman, Proc. Natl. Acad. Sci. U.S.A. 2006, 103, 15038-15043; c) K. H. Xu, X. Liu, B. Tang, ChemBioChem 2007, 8, 453-458; d) K. H. Xu, X. Liu, B. Tang, G. W. Yang, Y. Yang, L. G. An, Chem.-Eur. J. 2007, 13, 1411-1416.
[11] a) N. Umezawa, K. Tanaka, Y. Urano, K. Kikuchi, T. Higuchi, T. Nagano, Angew. Chem. Int. Ed. 1999, 38, 2899-2901; b) B. Song, G. L. Wang, M. Q. Tan, J. L. Yuan, J. Am. Chem. Soc. 2006, 128, 13442-13450.
[12] A. R. Lippert, G. C. V. De Bittner, C. J. Chang, Acc. Chem. Res. 2011, 44, 793-804.
[13] L. P. Du, M. Y. Li, S. L. Zheng, B. H. Wang, Tetrahedron Lett. 2008, 49, 3045-3048.
[14] B. C. Dickinson, C. Huynh, C. J. Chang, J. Am. Chem. Soc. 2010, 132, 5906-5915.
[15] a) E. W. Miller, A. E. Albers, A. Pralle, E. Y. Isacoff, C. J. Chang, J. Am. Chem. Soc. 2005, 127, 16652-16659; b) B. C. Dickinson, J. Peltier, D. Stone, D. V. Schaffer, C. J. Chang, Nat. Chem. Biol. 2011, 7, 106-112.
[16] J. L. Major Jourden, S. M. Cohen, Angew. Chem. Int. Ed. 2010, 49, 6795-6797.
[17] a) E. Sella, D. Shabat, Chem. Commun. 2008, 5701-5703; b) C. A. Blencowe, A. T. Russell, F. Greco, W. Hayes, D. W. Thornthwaite, Polym. Chem. 2011, 2, 773-790.
[18] F. Kratz, I. A. Muller, C. Ryppa, A. Warnecke, ChemMedChem 2008, 3, 20-53.
[19] H. Y. Lee, X. Jiang, D. W. Lee, Org. Lett. 2009, 11, 2065-2068.
[20] J. L. Major Jourden, K. B. Daniel, S. M. Cohen, Chem. Comm. 2011, 47, 7968-7970.
[21] C. De Gracia Lux, S. Joshi-Barr, T. Nguyen, E. Mahmoud, E. Schopf, N. Fomina, A. Almutairi, J. Am. Chem. Soc. 2012, 134, 15758-15764.
[22] F. Kielar, M. E. Helsel, Q. Wang, K. J. Franz, Metallomics 2012, 4, 899-909.
[23] G. C. Van de Bittner, E. A. Dubikovskaya, C. R. Bertozzi, C. J. Chang, Proc. Natl. Acad. Sci. U.S.A. 2010, 107, 21316-21321.
[24] M. S. T. Goncalves, Chem. Rev. 2009, 109, 190-212.
[25] E. Sella, A. Lubelski, J. Klafter, D. Shabat, J. Am. Chem. Soc. 2010, 132, 3945-3952.
[26] J. Zielonka, A. Sikora, M. Hardy, J. Joseph, B. P. Dranka, B. Kalyanaraman, Chem. Res. Toxicol. 2012, 25, 1793-1799.
[27] a) Y. Emre, C. Hurtaud, T. Nubel, F. Criscuolo, D. Ricquier, A. M. Cassard-Doulcier, Biochem. J. 2007, 402, 271-278; b) A. Mantovani, A. Sica, M. Locati, Eur. J. Immunol. 2007, 37, 14-16; c) A. P. West, I. E. Brodsky, C. Rahner, D. K. Woo, H. Erdjument-Bromage, P. Tempst, M. C. Walsh, Y. Choi, G. S. Shadel, S. Ghosh, Nature 2011, 472, 476-U543.
[28] B. Uttara, A. V. Singh, P. Zamboni, R. T. Mahajan, Curr. Neuropharmacol. 2009, 7, 65-74.

What is claimed is:

1. A compound having the formula: 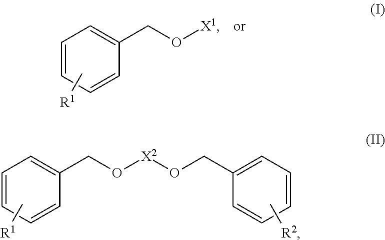
wherein,
R¹ and R² are independently —B(OH)₂ or 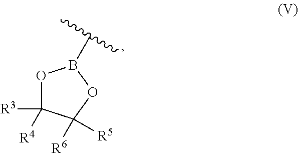
wherein,
R³, R⁴, R⁵ and R⁶ are independently hydrogen, halogen, —N₃, —NO₂, —CF₃, —CCl₃, —CBr₃, —CI₃, —CN, —OH, —NH₂, —COOH, —CONH₂, —SH, —SO₃H, —SO₄H, —SO₂NH₂, —NHNH₂, —ONH₂, —OCH₃, —NHC(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
X¹ is a monovalent pro-fluorophore moiety; and
X² is a divalent pro-fluorophore moiety.

2. The compound of claim 1, wherein R¹ and R² are independently

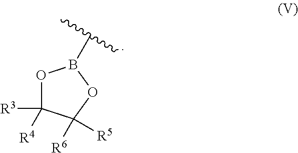

3. The compound of claim 2, wherein R¹ and R² are identical.

4. The compound of claim 3, wherein R³, R⁴, R⁵, and R⁶ are independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

5. The compound of claim 4, wherein R³, R⁴, R⁵ and R⁶ are independently substituted or unsubstituted alkyl.

6. The compound of claim 5, wherein R³, R⁴, R⁵ and R⁶ are methyl.

7. The compound of claim 1, wherein the compound of formula (I) has the formula:

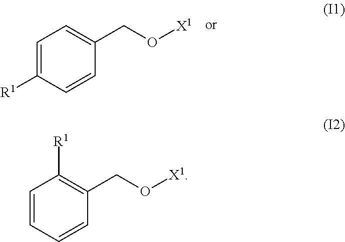

8. The compound of claim 7, wherein,
X¹ is a monovalent pro-fluorescein, monovalent pro-coumarin, monovalent pro-dansyl, monovalent pro-bimane, monovalent pro-eosin, monovalent pro-rhodamine, monovalent pro-cyanine, monovalent nile red, monovalent xanthone, monovalent xanthene, monovalent flazo orange, monovalent Snarf1, or monovalent resorufin, including conjugates, derivatives and analogues thereof.

9. The compound of claim 8, wherein X¹ is monovalent pro-fluorescein or monovalent pro-coumarin.

10. The compound of claim 1, wherein the compound of formula (II) has the formula:

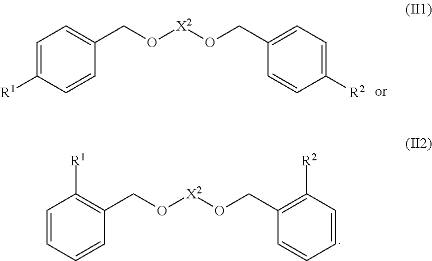

11. The compound of claim 10, wherein,
X² is a divalent pro-fluorescein, divalent pro-coumarin, divalent pro-dansyl, divalent pro-bimane, divalent pro-eosin, divalent pro-rhodamine, divalent pro-cyanine, divalent nile red, divalent xanthone, divalent xanthene, divalent flazo orange, divalent Snarf1, or divalent resorufin, including conjugates, derivatives and analogues thereof.

12. The compound of claim 11, wherein X² is divalent pro-fluorescein.

13. The compound of claim 1 having the formula:

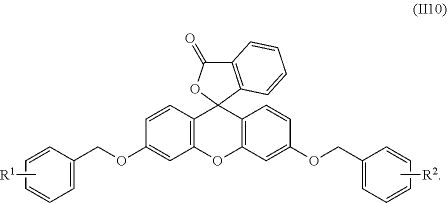

14. The compound of claim 13 having the formula:

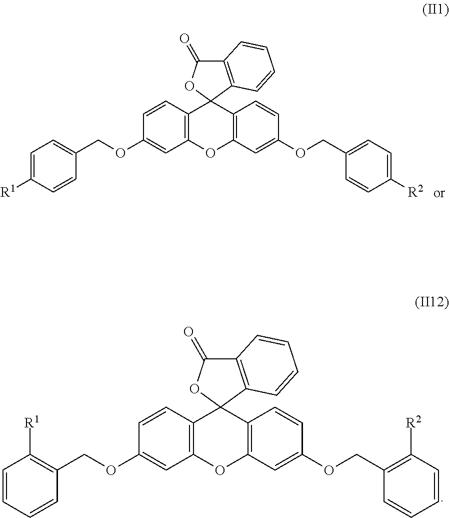

15. The compound of claim 14 having the formula:

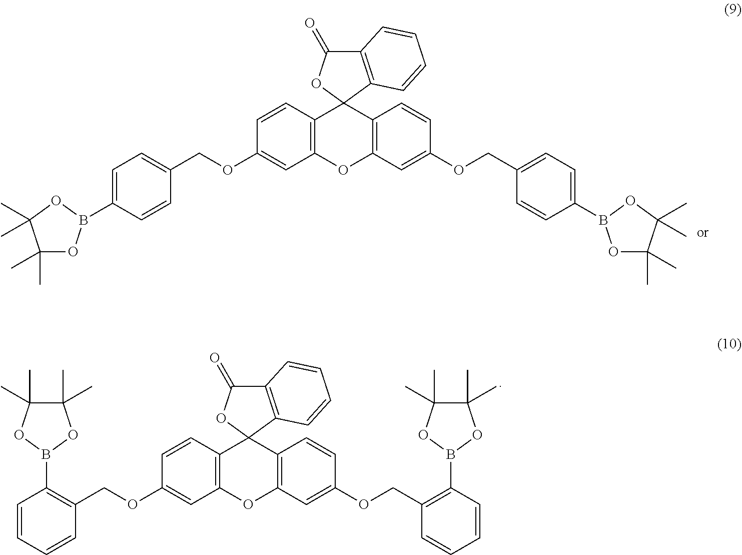

16. The compound of claim 1 having the formula:

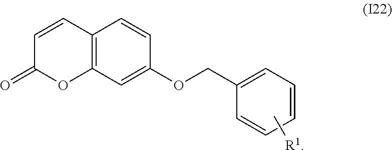

17. The compound of claim 16 having the formula:

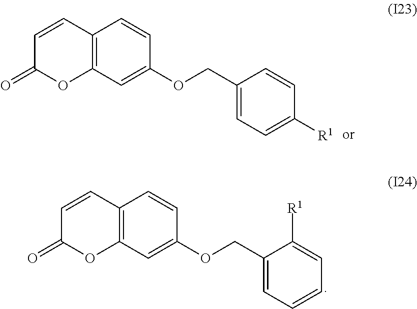

18. The compound of claim 17 having the formula:

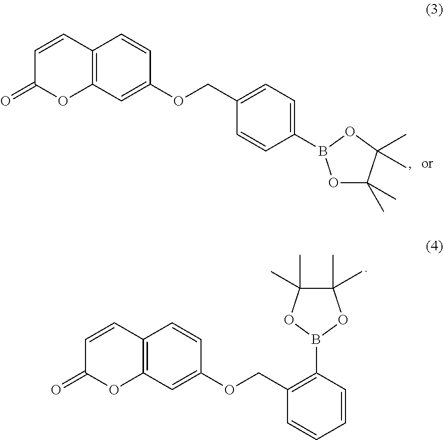 [SMILES: CC1(C)OB(c2ccc(COc3ccc4ccc(=O)oc4c3)cc2)OC1(C)C ; CC1(C)OB(c2ccccc2COc2ccc3ccc(=O)oc3c2)OC1(C)C]

19. The compound of claim 1, wherein R¹ and R² are —B(OH)₂.

20. A method of detecting a ROS compound, said method comprising:
(i) contacting a compound of formula (I) or formula (II) with a ROS compound;
(ii) allowing said ROS compound to react with said compound of formula (I) thereby removing R¹ or allowing said ROS compound to react with said compound of formula (II) thereby removing R¹ and R² thereby forming a fluorescent compound;
(iii) detecting said fluorescent compound, thereby detecting said ROS compound.

21. The method of claim 20, wherein said ROS compound is H₂O₂, OCl⁻, or ONOO⁻.

22. The method of claim 20, wherein R¹ and R² are independently

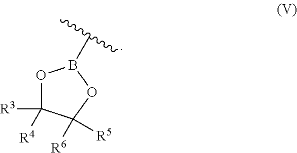

23. The method of claim 22, wherein R³, R⁴, R⁵ and R⁶ are independently substituted or unsubstituted alkyl.

24. The method of claim 20, wherein
X¹ is monovalent pro-fluorescein or monovalent pro-coumarin; and
X² is divalent pro-fluorescein.

25. A method for detecting a ROS compound in vivo in a subject, said method comprising:
(i) administering to a subject, a compound of formula (I) or formula (II);
(ii) allowing said compound of formula (I) to react with a ROS compound, thereby removing R¹ or allowing said compound of formula (II) to react with a ROS compound thereby removing R¹ and R² thereby forming a fluorescent compound;
(iii) detecting said fluorescent compound, thereby detecting said ROS compound in vivo.

26. The method of claim 25, wherein R¹ and R² are independently

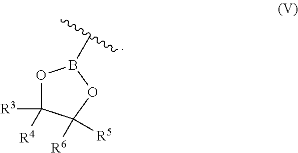

27. The method of claim 26, wherein R³, R⁴, R⁵ and R⁶ are independently substituted or unsubstituted alkyl.

28. The method of claim 25, wherein
X¹ is monovalent pro-fluorescein or monovalent pro-coumarin; and
X² is divalent pro-fluorescein.

29. The method of claim 25, wherein R¹ and R² are removed at physiological levels of H₂O₂.

30. The method of claim 29, wherein R¹ and R² are removed at a concentration of about 5 μM to about 100 μM of H₂O₂.

31. The method of claim 25, wherein said detecting comprises detecting the location of said ROS compound in said subject.

32. The method of claim 25, wherein said detecting comprises quantifying an amount of said ROS compound in said subject.

33. The method of claim 32, wherein said monovalent pro-fluorophore moiety and said divalent pro-fluorophore have substantially no fluorescence in the absence of said ROS compound.
